(19) United States
(12) Patent Application Publication
(10) Pub. No.: US 2019/0074093 A1
(43) Pub. Date: Mar. 7, 2019

(54) Management System and Method for a Caregiver Support Platform

(71) Applicant: Cariloop, Inc., Dallas, TX (US)

(72) Inventors: Michael Walsh, Dallas, TX (US); Peter Taliancich, Frisco, TX (US); Dane Roper, Dallas, TX (US); Jason Osburn, Plano, TX (US)

(73) Assignee: Cariloop, Inc., Dallas, TX (US)

(21) Appl. No.: 16/122,792

(22) Filed: Sep. 5, 2018

Related U.S. Application Data
(60) Provisional application No. 62/554,431, filed on Sep. 5, 2017.

(51) Int. Cl. G16H 80/00; G16H 40/20; G16H 10/20

(52) U.S. Cl. CPC G16H 80/00; G16H 10/20; G16H 40/20

(57) ABSTRACT

The management system and method for the caregiver support platform includes healthcare coaching services and a digital case management portal. The support platform is developed following a risk assessment survey assessing likelihood of an individual becoming a caregiver. A support coach works with the caregiver to determine what is needed in a support platform. The support coach reviews case details, analyzes resources available to the caregiver, and prepares a care plan based on the options preferred by the caregiver.

### CROSS REFERENCE TO RELATED APPLICATIONS

This application claims priority to U.S. Provisional Patent Application No. 62/554,431, filed Sep. 5, 2017.

### STATEMENT REGARDING FEDERALLY SPONSORED RESEARCH OR DEVELOPMENT

Not applicable.

### TECHNICAL FIELD OF INVENTION

This invention relates to a management system and a for a caregiver support platform.

### BACKGROUND OF THE INVENTION

Caregiving touches virtually every one of us at some point in our lives, from childbirth to eldercare. Many in our nation's workforce are well acquainted with the daily challenges of caregiving. The problem is far-reaching enough that even employers are heeding the call to play a role in helping make life easier for their working caregivers.

In the United States, an estimated 43.5 million adults—nearly 20 percent of the adult population—provide unpaid caregiving to an adult or child. A caregiver is defined as an unpaid spouse, partner, family member, friend, or neighbor who plays a critical role in helping a loved one with daily activities and/or medical tasks.

Almost everyone in the workforce will come face-to-face with the challenges of caregiving at some point—some just may not realize it yet. They may be caring for a loved one now, or have in the past, or may anticipate needing to serve in this role one day. It's an issue that can spring up overnight and last for years. From sudden health emergencies to chronic diseases, caregiving eventually touches us all in one way or another.

There are legions of dedicated employees across the country working fulltime jobs who uproot their personal lives and careers to provide care, support and affection to those in need—for a newborn, a sick child, an adult family member with a life-limiting disease, a sibling, an aging parent or grandparent, or a friend. For many, caregiving is comparable to holding down a second job, and the lines between work and personal lives become blurry, at best, when the care of a loved one is top-of-mind. Tethered by an emotional struggle to leave unpaid caregiving at home, these people must go to work and are expected to perform at the highest level. Caregiving disrupts virtually every aspect of an employee's day-to day activities and job responsibilities, and can include: preparing meals; providing personal care; handling finances; managing medications, helping with bathing and dressing—and more.

Today, the scope of caregiving has broadened for working Americans who are often tasked with varied, intermittent responsibilities to help navigate health coverage, drive to doctors' appointments, participate in therapy sessions, identify assisted living or alternative housing options, or provide other “behind the scenes” support. Even when they are at work, caregivers can feel distracted by the growing and often unexpected list of duties that require timely attention or await them at home. They are often forced to take time off from work and careers to accomplish everything that needs to get done. Caregiving affects employees on a daily basis, with some workers devoting 20+ hours a week to their caregiving responsibilities. Many are also spending thousands of dollars in out-of-pocket expenses and performing nursing care duties with little to no training.

Many people consider it an honor to provide caregiving, and in many instances, these individuals sacrifice work/life balance and risk losing their jobs. Among caregivers who are employed or who have been employed during their time as caregivers, some have had to reduce their hours or job responsibilities, take leave from their positions, quit their jobs or retire early to accommodate their caregiving duties.

Medical advances are extending lives, hospital stays are shorter, families are smaller, and society is more mobile. More women are working, and baby boomers are working past retirement, while others are re-entering the labor force in their 50s, 60s and 70s. This means fewer family members are available in the home to provide caregiving as needs arise.

U.S. companies that fail to address these issues pay the price: businesses lose between $17.1 billion and $33.6 billion annually on lost productivity, depending on the level of caregiving involved. That equals $2,110 for every full-time worker who is a caregiver for an adult. They also face higher health insurance costs. This does not take into account the number of promotions or assignments employees turn down that require travel or relocation away from their caregiving responsibilities.

Millennial (born 1979-2000) and Generation X (born 1965-1978) caregivers are more likely to be employed than Baby Boomer (born 1946-1964) and Mature (born before 1946) caregivers—and Matures and Baby Boomers are more likely to be retired. Among caregivers who are employed, however, Millennials are more likely to have experienced some form of adverse action taken by their employers as a result of their caregiving.

For employers wondering which of their employees are currently or may soon become caregivers, the answer is simple: All of them. It's not a question of if, but when.

The toll of caregiving on corporate America is astounding: 49% arrive to their place of work late, leave early or take time off, while 15% take a leave of absence, and 14% reduce their hours or take a demotion. 7% receive a warning about performance/attendance, 5% turn down a promotion, 4% choose early retirement, 3% lose job benefits, and 6% give up working entirely. 69% of working caregivers caring for a family member or friend report having to rearrange their work schedule, decrease their hours, or take an unpaid leave in order to meet their caregiving responsibilities.

In terms of employee sacrifices, sixty-one percent of caregivers experience at least one change in their employment due to caregiving, such as cutting back work hours, taking a leave of absence, and/or receiving a warning about performance/attendance. While the vast majority of caregivers (87%) are caring for a family member, the family relationship varies by generation. For instance, 21% of Millennials are more likely to be caring for a grandparent compared to Generation X (9%), Baby Boomers (1%) and Matures (none).

33% of Millennials have reduced their hours or job responsibilities. 24% of Millennials and 17 percent of Generation X have taken a leave of absence. 17% of Baby Boomers have retired early/quit their job. For the nation's caregivers, the experience is emotionally draining, physically exhausting, and life-altering. In fact, about one in ten (11%) of caregivers report that caregiving has caused their physical health to get worse. Primary caregivers (22%) are more likely than non-primary caregivers (10%) to say their financial situation has worsened since becoming a caregiver.

Eldercare, alone, costs businesses $6.6 billion to replace employees (9 percent left work either to take early retirement or quit). Nearly $6.3 billion in workday interruptions (coming in late, leaving early, taking time off during the day or spending work time on eldercare matters). $5.1 billion in absenteeism.

While employee caregivers face challenges that lead to such high absenteeism and workday interruption costs, only: 53% of employers offer flexible work hours/paid sick days; 32% offer paid family leave; 23% offer employee assistance programs and; 22% allow telecommuting regardless of employee caregiving burden.

Fortunately, a growing number of compassionate, forward-thinking public and private sector employers have taken steps to support their employee caregivers by offering paid leave or other programs. But more needs to be done.

A national strategy is falling into place with the January 2018 passage of the bipartisan Recognize, Assist, Include, Support and Engage (RAISE) Family Caregivers Act. RAISE requires the U.S. Secretary of Health and Human Services (HHS) to develop, maintain and update an integrated national strategy to support family caregivers. The idea behind this legislation is that caregivers are the backbone of the nation's care system and encourages employers to make it easier for them to coordinate care for their loved ones, get information and resources, and take a break so they can rest and recharge.

Paid leave policies in a handful of states are also motivating millions of workers and their families to cope with caregiver responsibilities. As of January 2018, five U.S. states and the District of Columbia have enacted laws guaranteeing paid family and medical leave: California, New Jersey, Rhode Island, New York, and Washington. These programs provide workers with a share of their wages when they need time to care for a family member with a serious health condition, bond with a new child or deal with their own serious medical issue. In California, for example, an analysis found that the program increases the short-term and long-term labor force participation rates of family caregivers with an eight percent increase in the short run and a 14 percent increase in the long run.

Employee caregivers need time to care for a loved one without jeopardizing their good standing at work. They also need supportive services to help navigate the complex decisions required when caregiving. The success of current programs at leading companies demonstrates that progress is possible—and that there are effective, affordable and proven models that offer options for families, businesses and economies. Ultimately, caregivers make great workers. They are strong, dedicated, resourceful and, when treated with respect and compassion, loyal.

As more companies begin to understand the impact of an aging U.S. population and the concomitant pressures on their employees to provide care, the companies acknowledge that the employees need. According to the Families and Work Institute's 2016 National Study of Employers, 42 percent of employers offer elder care resource and referral services and 78 percent provide some amount of either paid or unpaid time off for employees who need to care for aging relatives.

The idea is not to simply spend more money and offer more benefits, but what is needed is an innovative new way to support employee caregivers. By incorporating community resources and taking advantage of emerging business platforms and innovative programs, leaders can more effectively create a cultural shift that celebrates and supports the lives of its employees—especially those who sacrifice so much to care for the ones they love.

The computer resources available to address this problem are ineffective and can be overwhelming to an already overtaxed caregiver. Internet searches for home health care, transportation services, prescription programs, assisted living or nursing facilities, or any of the other caregiver tasks, will reveal thousands of individual service providers requiring many hours to review and analyze. Sorting through the information available to find the best options for a loved one is a daunting and time-consuming task. Even with careful searching, potentially good options may be missed without input from a knowledgeable source. There exists a need to assist caregivers with the task of locating and evaluating resources and engaging necessary services for a loved one.

### SUMMARY OF THE INVENTION

The present invention is a management system and method for a caregiver support platform with experienced coaches to assist in the caregiving process. The system is a tech-enabled Caregiver Support Platform which guides caregivers as they plan for and manage care of a loved one, allowing caregivers to securely collaborate across electronic devices to share medical, financial and legal documents, and provide access to a support coach during the process.

The management system and method for the support platform includes healthcare coaching services and a digital case management portal. The support platform is developed for individual caregivers following a risk assessment using a risk assessment survey that assesses the risk or likelihood of an individual becoming a caregiver for a family member or close friend. Risk assessment and risk assessment survey may also be referred to as a caregiver risk assessment, caregiver risk assessment survey, or survey.

When a caregiver creates an account, a support coach) works with the caregiver to determine what is needed in a support platform. The support coach will review the case details, analyze the resources available to the caregiver, and prepare a care plan based on the selection of options preferred by the caregiver. The support coach may also be referred to as a coach, care coach, healthcare support coach, healthcare coach, instructor, teacher, counselor, or guidance counselor.

The present invention is management system and method for a caregiver support platform comprising: a computer processor having subprograms operating on the computer processor, having the computer processor coupled to one or more input/output devices, one or more input/output ports for receipt and transmission of data, requests, replies and commands, and having a temporary memory for storage of data including caregiver support platform data, caregiver support platform resources, and administrative data. One or more database memory is coupled to the computer processor, so that the one or more database memory stores data and commands associated with operation of the subprograms including caregiver account data, caregiver support platform data, caregiver support platform resources, and administrative data.

The management system and method for the caregiver support platform has an evaluation subprogram operated through the management system that executes a survey program with a first user to assess the first user's present status and potential risk of becoming a caregiver to another person. The evaluation subprogram assigns a first caregiver level to the first user based on the first user's input to the survey program and the evaluation subprogram transfers data input by the first user in response to said survey program into said computer processor and database memory.

The management system and method for the caregiver support platform has an account creation subprogram operated on the management system that executes an account creation program to create a first care case on the management system associated with the first user based on the first caregiver level assigned to said first user, the account creation subprogram requesting and receiving information from the first user to be associated with the first user's account. The account creation subprogram maintains information that can support the first user in the first care case.

The management system and method for the caregiver support platform has a coaching subprogram operated on the management system that executes a coaching program that can be accessed by a support coach to support the entry and maintenance of data information and resources from the management system that can assist the first user, and also has a notification system subprogram operated on the management system that transmits messages to the first user based on requests, commands, responses or events related to the first user.

The management system for the caregiver support platform and method further has an administrative subprogram operated on the management system that executes an administrative program to input and maintain data relating to the first user, including information and resources relating to the first user's status as a caregiver. The management system for the caregiver support platform has an interactive dashboard display driven by the management system having customized displays of information directed to the first user, one or more other users, one or more support coaches, or one or more administrative personnel, and the management system allows access to information maintained on the management system and displayed on the interactive dashboard display based on display rights associated with said first user, one or more other users, one or more support coaches, or one or more administrative personnel.

The evaluation subprogram operated on the management system and method for the caregiver support platform executes the survey program through an external facing portion of the management system and provides the first user with an option to create an account after completion of the survey and evaluation. The account creation subprogram operated on the management system communicates to the first user through an external facing portion of the management system to create the first user's account having the first care case associated with the first user as a primary caregiver and account owner and enters data from the first user as a primary caregiver on a caregiver portal, with the data being received into said management system. A portal may also be alternatively referred to as input/output portals, input/output ports, ports, input/output to the management system and method of the present invention. Portals may also be specifically referenced as caregiver portals, member portals, user portals, personal portals, coach portals, coaching portals, coach view portals, admin portals. care portals, case portals, caregiver support system portals, digital case management portals, management system portals, management portals, and organization portals.

The coaching subprogram operated on the management system and method communicates to the first user through an internal facing portion of the caregiver support platform to support the input of data into the support coach portal, access data, information and resources maintained on the management system, and output data and information from the management system.

The management system and method for the caregiver support platform further has an analytics protocol operated on the management system that analyzes the first user data input for the first care case account established for the first user and maintains personnel data related to the support coach assigned to the first user.

The support coach assigned to the first user accesses data related to the first user, caregiver support platform information, and resources to prepare a care plan specific to the needs of the first user and provide one or more caregiver support coaching sessions to the first user with a transmission of output information to the first user. The first care case can be accessed by the first user, and includes data and information including the care plan prepared by the support coach, audio and video discussions of coaching sessions with the first user, notifications from the management system, and resources made available to the first user from caregiver support system data repository.

Upon account creation, the notification system subprogram transmits an automated message to the first user as the primary caregiver confirming the establishment of an account, an automated message to one or more support coaches assigned to the first user's care case, and an automated message to one or more administrative personnel reporting the first user's caregiver account creation. A care team is established that comprises the first user as the primary caregiver, one or more support coaches, and one or more additional caregivers authorized by the primary caregiver. Persons assigned to the care team are authorized by the first user as the primary caregiver to access the care case using management system portal to input documents, discussions, participate in coaching sessions and review stored coaching sessions.

The external facing and internal facing subprograms of the management system and method for the caregiver support platform may be accessed from, or input into, the computer processor using a desktop computer, mobile phone, intelligent pad device, or other personal communication device.

The management system and method for the caregiver support platform further has analytics engine to track, analyze, and report caregiver support platform interactions and events, survey responses, caregiver accounts, care case needs and discussions, and account and case relationships.

### BRIEF DESCRIPTION OF THE DRAWINGS

FIG. 1 is a view of a system for caregiver data in accordance with the present invention.

FIG. 2 is an overview of the system steps.

FIG. 3 is a flowchart of the risk assessment decision tree for an individual user.

FIG. 4 is a flowchart of the risk assessment decision tree for an organization.

FIG. 5 is an illustrative example of the interaction between the caregiver dashboard and tabs.

FIG. 6A is an illustrative example of the interaction between the admin dashboard and tabs. FIG. 6B is an illustrative example of another embodiment of the interaction between the admin dashboard and tabs.

FIG. 7B is an illustrative example of the interaction between the support coach dashboard and tabs. FIG. 7B is an illustrative example of another embodiment of the interaction between the coach dashboard and tabs.

FIG. 8 is an illustrative example of the caregiver user interaction environment with the support platform system.

FIG. 9A-B is an illustrative example of workflow diagram illustrative of operations and data generation of a caregiver support platform and dashboard user interface data to view on a dashboard user interface.

### DETAILED DESCRIPTION

The present invention is a management system and method for a caregiver support platform with experienced support coaches to assist in the caregiving process. The system is a tech-enabled Caregiver Support Platform which guides caregivers as they plan for and manage care of a loved one, allowing caregivers to securely collaborate across electronic devices to share medical, financial and legal documents, and provide access to a support coach during the process.

The management system and method for the support platform includes healthcare coaching services and a digital case management portal. The support platform is developed for individual caregivers following a risk assessment using a risk assessment survey that assesses the risk or likelihood of an individual becoming a caregiver for a family member or close friend. Risk assessment and risk assessment survey may also be referred to as a caregiver risk assessment, caregiver risk assessment survey, or survey.

When a caregiver creates an account, a support coach) works with the caregiver to determine what is needed in a support platform. The support coach will review the case details, analyze the resources available to the caregiver, and prepare a care plan based on the selection of options preferred by the caregiver. The support coach may also be referred to as a coach, care coach, healthcare support coach, healthcare coach, instructor, teacher, counselor, or guidance counselor.

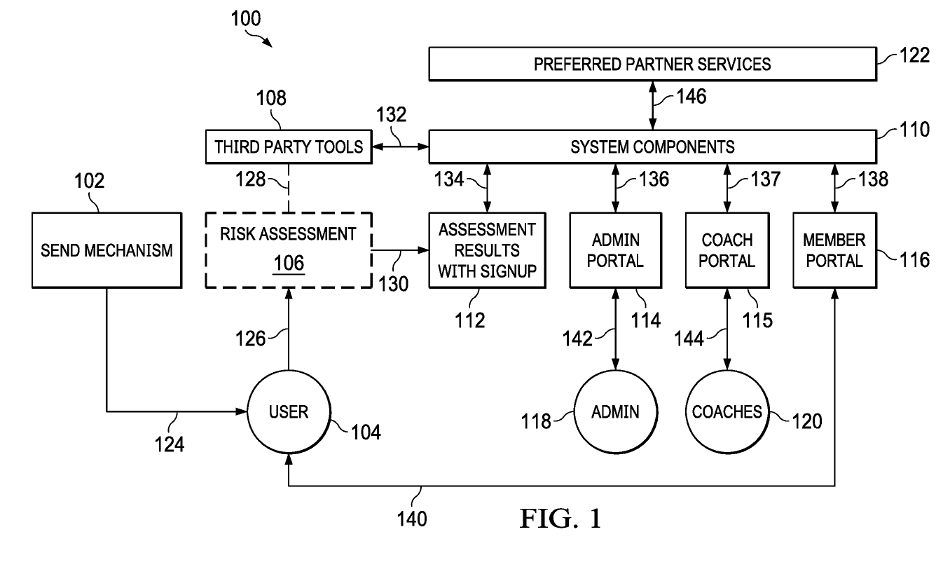

FIG. 1 shows an overview 100 of the management system and method of the caregiver support platform. A send mechanism 102 is the initial contact for a Caregiver user. Various send mechanisms may utilized, for example the initial contact may be by email, text message, social media contact or by a user directly contacting the marketing website for the caregiver support platform. An email or text message send mechanism may be generated by an employer organization or a by third party marketing tool 108.

The send mechanism 102 of the management system of the present invention will direct 124 a user 104 to link to the risk assessment survey 106. The risk assessment survey 106 is facilitated 128 by a third party 108 data collection tool and data generated by the survey is transferred 132 to one or more of the system components 110. The risk assessment survey can also be facilitated on one or more of the system components 110 of the management system of the caregiver support platform. At the completion of the survey 106, the user 104 submits 130 the survey to view the assessment results 112 and has the option to set up an account.

If the option to set up an account is selected, the user 104 inputs 134 account information to the management system 110 and an account is created. The user 104 is then directed to log-in 140 to the member portal 116 to access 138 resources in the support platform system 110. Notifications regarding the account creation may be automatically sent to the user 104 from the support platform system 110. The user (or primary caregiver), is the account owner and has established rights to access the care case.

A support coach 120 is assigned to the case and notified by the management system of the support platform system 110 of the assignment. The support coach 120 logs-in 144 to the coach portal 115 to access 137 the support platform system 110. The support coach 120 contacts the user 104 to provide support and prepare a care plan, which may include facilitating a user 104 to link 146 to preferred partner services 122 based on the user's 104 specific needs.

Administrators 118 log-in 142 to the Admin portal 114 and accesses the components of the management system of the caregiver support platform 110 for management of accounts, personnel and data.

The management system of the caregiver support platform 110 encompasses a digital case management portal that stores case data, coach-caregiver discussions, audio or video recordings of coaching sessions, case documents uploaded by the caregiver or support coach, and customized research for care support plans. The digital case management portal provides an access point for the caregiver and the support coach to interact with discussions about care plan needs, service provider suggestions specific to the case, and steps for managing the care plan. Caregivers who are account holders can share access to the case portal with family members and care team members to facilitate collaboration on the support platform. The case portal is accessible by web-enabled devices, such as desktop or laptop computers, tablets, mobile phones, and similar devices.

The management system of the caregiver support platform has an external-facing portion with a Member or user Portal for access by Caregivers, and an internal facing portion with Coaching and Admin portals for access by support coaches, assistant coaches and administrators.

On the external-facing Member Portal side, Caregivers and care team members can interact with the support coach via written message, audio coaching, or video-chat sessions, start new discussions or review previous discussions, upload or review documents; review the care plans and related research, access previously recorded coaching sessions, access services from preferred providers suggested by the support coach, and share the case with family or care team members.

The internal facing portal side of the management system of the caregiver support platform is accessible by administrators 118, assistant coaches and support coaches 120. The administrator (Admin) role is restricted to executive level personnel and has the ability to view all cases/notes, create coaches, assign support coaches, and related administrative activities. Assistant coaches can view case details and assign support coaches. Support coaches can view, work on and add/modify content for assigned care cases.

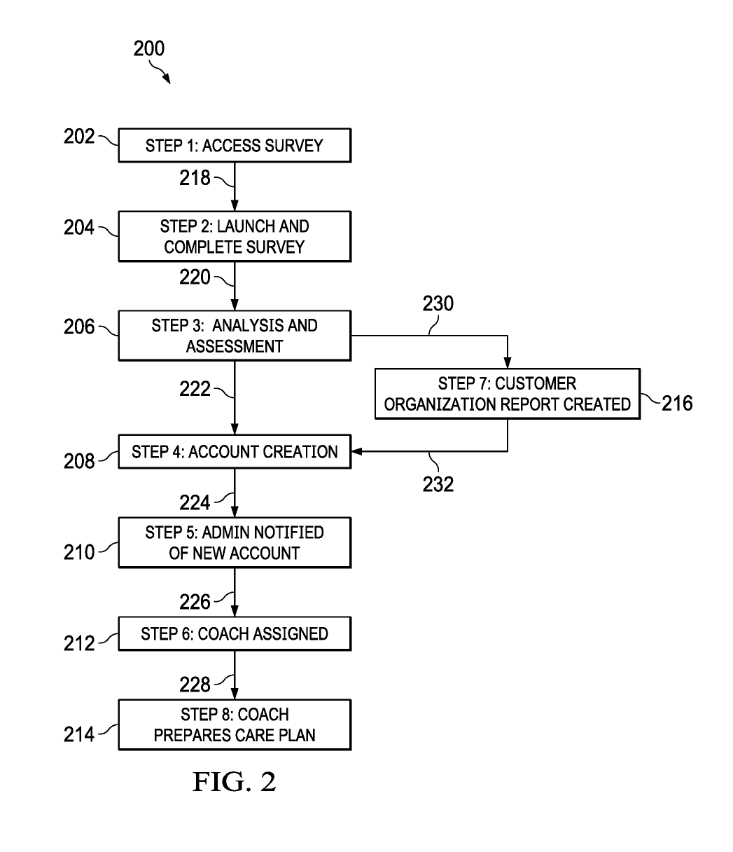

FIG. 2 shows eight basic steps 200 utilized in the management system and method of the caregiver support platform. Steps 1-6 and 8 are applicable to all Users who are setting-up accounts. Steps 1, 2 and 4 are User operated on the external facing portion of the platform. Steps 3, 5, 6 and 8 are operated on the internal facing side of the platform for all cases. Step 7 is an additional step utilized when a Customer Organization is offering the service to employees as part of a corporate benefits program and is operated on the internal facing side of the platform. The basic steps are as follows:
Step 1 (202): User accesses Caregiver Risk Assessment on the website;
Step 2 (204): Caregiver Risk Assessment is launched and the User answers questions;
Step 3 (206): Answers are analyzed and a risk status is presented to User;
Step 4 (208): User is given option to create an account:
Step 5 (210): Administrator is informed of a new case requiring action; e.g. assign support coach;
Step 6 (212): Administrator assigns support coach or support coach is assigned by an automated process, and support coach and User are notified;
Step 7 (216): For Customer Organizations, a Caregiver Risk Assessment Report is created; and
Step 8 (214): Support coach suggests services for caregiver support platform.
   Each step is described in more detail in the following paragraphs.

Step 1 (202): User Accesses the Caregiver Risk Assessment

A User accesses the Caregiver Risk Assessment survey through the management system and method of the caregiver support platform by either directly accessing the website or a User may be directed to the Caregiver Risk Assessment by a link sent via email, social media message, mobile phone text message, or other direct contact means.

A User who is an employee of a Customer Organization may receive the link to the management system of the caregiver support platform in a communication from within the Customer Organization, such as from the Benefits team or Human Resources department; or the Customer Organization may provide the employee email addresses to a marketing platform provider for direct contact. After Step 1 (202) contact, the user is directed 218 to Step 2 (204).

Step 2 (204): Caregiver Risk Assessment is Launched and the User Answers Questions

The Caregiver Risk Assessment of the management system and method of the caregiver support platform is launched and the User answers questions to determine their Caregiver risk/status. The decision-tree style-questions are presented to the User based on answers to previous questions. Users are taken through questions regarding caregiving activities, tasks performed for loved ones and the impact caregiving has on the user's time and resources. Further questions ask if a user anticipates being a caregiver in the future and ask about how prepared a user is to be caregiver. Answers are supplied by checking the appropriate boxes. After completion of the survey in Step 2 (204), the user submits 220 the survey and is provided with results in Step 3 (206).

Step 3 (206): Answers are Analyzed and a Risk Status is Presented to User.

After the User's answers are submitted and analyzed by the management system and method of the caregiver support platform, one of several Risk Assessments are provided based on the User's answers to the questions on the Risk Assessment survey. Possible Risk Assessments are: High-Risk Caregiver (presently a caregiver), Moderate-Risk Prepper (anticipates being a caregiver), Low-Risk caregiver (well-prepared or possible emergency caregiver), and Non-Caregiver.

Individual Users assessed in Step 3 (206) as high or moderate risk are given the option 222 of creating an account in Step 4 (208) by the management system and method of the caregiver support platform. If the user is associated with a customer organization, following Step 3 (206), additional Step 7 (216), as described below, is activated 230 to generate a report for the organization prior to account creation. After the Caregiver Risk Assessment Report has been reviewed with the Customer Organization, users associated with the organization are directed 232 to the account creation steps.

Step 4 (208): User is Given Option to Create an Account

If the assessment of the management system of the caregiver support platform determines that a User is a current caregiver (high risk) or has a near-future (moderate) risk for being a Caregiver, the user is given by the management system and method of the caregiver support platform the option to set up an account and access the Member portal of the external-facing User portion of the caregiver support platform via a “Create Account” button that, once clicked, will present fields to enter information required to create an account. On the account creation screen, the User will be prompted by the management system and method of the caregiver support platform to enter the name of the loved-one, and information about the biggest caregiving challenge. The User will set and confirm a password to access the account. After creation of an account in Step 4 (208) by the management system of the caregiver support platform, a notification 224 alerts Admin that a new account has been created. Users are also referred to a members, caregivers and caregiver users.

Step 5 (210): Administrator is Informed of a New Case Requiring Action; e.g. Assign Coach

When an account is created by the management system and method of the caregiver support platform, the Assessment results are saved to the case on the internal-facing portion of the caregiver support platform, the User is added to a Customer Relationship Management (CRM) account as an individual member or is associated with a Customer Organization as an employee, and a notification is sent via the internal facing caregiver support platform to inform the Administrator that a new case has been created requiring action, e.g. a support coach needs to be assigned to the case. Support coaches may also be automatically assigned by the management system of the caregiver support platform based on an analysis by the caregiver support platform of the care case details and coaching personnel capabilities. Email(s) may also be sent by the management system of the caregiver support platform to the new member confirming and/or communicating important information about their membership, including notification that a support coach will be or has been assigned to the case. If a support coach is not automatically assigned by the management system of the caregiver support platform, after Admin is notified of the new account in Step 5 (210), a support coach will be assigned and notified 226 of the assignment.

Step 6 (212): Administrator Assigns Coach, and Coach and User are Notified

The Administrator of the management system and method of the caregiver support platform takes relevant action on the internal-facing portion of the Caregiver Support Platform, e.g. assigns a support coach to the case at which point the support coach is notified via the Support Platform messaging that they have been assigned to a case. Support coaches may also be automatically assigned by the management system of the caregiver support platform to the case following an analysis by the management system of the caregiver support platform. The Member is notified via the Support Platform messaging that a support coach has been assigned and contact information for the support coach is provided to the Member.

If the user is associated with a customer organization, following Step 3 (206), additional Step 7 (216) is activated 230 by the management system of the caregiver support platform to generate a report for the organization.

Step 7 (216): For Customer Organizations, a Caregiver Risk Assessment Report is Created

If a Customer Organization is offering the caregiver support platform as a service to employees, an email notification of the Caregiver Risk Assessment Survey will be sent to the employees. One or more employees will take the survey and submit results to receive a risk assessment. When a sufficient number of Risk Assessments (a minimum of 10-20%) have been completed, the Customer Support team compiles the results into illustrative charts and graphs, creates a Caregiver Risk Assessment Report, and reviews results with the Customer Organization.

The Caregiver Risk Assessment Report provides the Customer Organization with the following analytical documents: General Assessment Overview, Analysis of High Risk Employee Caregivers, Analysis of Moderate Risk Employee Caregivers, Caregiver Risk Summary, and Next Steps.

General Assessment Overview: The overview details the number of employee participants and the Employee Caregiver Risk Levels (high, moderate or low) among the participants. A pie chart is provided showing a breakdown of assessment participants by percentages as caregivers, preppers, perfectly prepared and non-caregivers.

Analysis of High Risk Employee Caregivers: This analysis provides bar charts showing the most common (high-level) caregiving tasks and the most common (day to day) caregiving tasks of the employee caregivers identified as high risk. Pie charts demonstrate mental wellness indicators, i.e., how frequently the employee caregivers worries about a loved one, financial wellness indicators, i.e., the percentage of employee caregivers who are providing financial support or a loved one's care, and emotional wellness indicators, i.e., the percentage of caregivers who are spending less time at work or with family and friends due to caregiving tasks.

Analysis of Moderate Risk Employee Caregivers: The analysis provides a pie chart demonstrating employee's preparedness to manage a health emergency and legal readiness for caregiving, i.e., having legal documents, such as Medical Powers of Attorney and Living Wills, prepared and accessible.

Caregiver Risk Summary: This summary rates an organization's overall level of caregiver risk. A summary of the high risk and moderate risk employee caregiver analyses is provided along with employee wellness areas impacted.

Next Steps: The Customer Organization is provided with suggestions for opportunities for improvement and engagement opportunities to implement the program.

After the Caregiver Risk Assessment Report has been reviewed with the Customer Organization, users associated with the organization are directed to the account creation step, and a coach will be assigned, as described above.

Step 8 (214): Coach Suggests Services for Caregiver Support Platform

The support coach assigned to the case by the management system and method of the caregiver support platform interacts with the user 220 and prepares a care plan responsive to the caregiver's needs. Based on Assessment responses, support coaches and/or an automated artificial intelligence (AI) coach assistant of the management system of the caregiver support platform can suggest to the User services provided by Preferred Partners that have been integrated into the caregiver support platform. The artificial intelligence coach assistant may also be referenced as an AI coach assistant, AI assistant, automated coach assistant, or coach assistant.

A support coach is able to suggest services including transportation services, prescription medication services, location and review of care facilities, and other services that meet a user's caregiving needs. For example, a support coach is able to suggest providers of transportation services if there is need to get a loved one to a medical appointment, or the support coach can provide information about assisted living facilities if a caregiver needs to find residential care for a loved one.

Caregiver Assessment for Individual Users

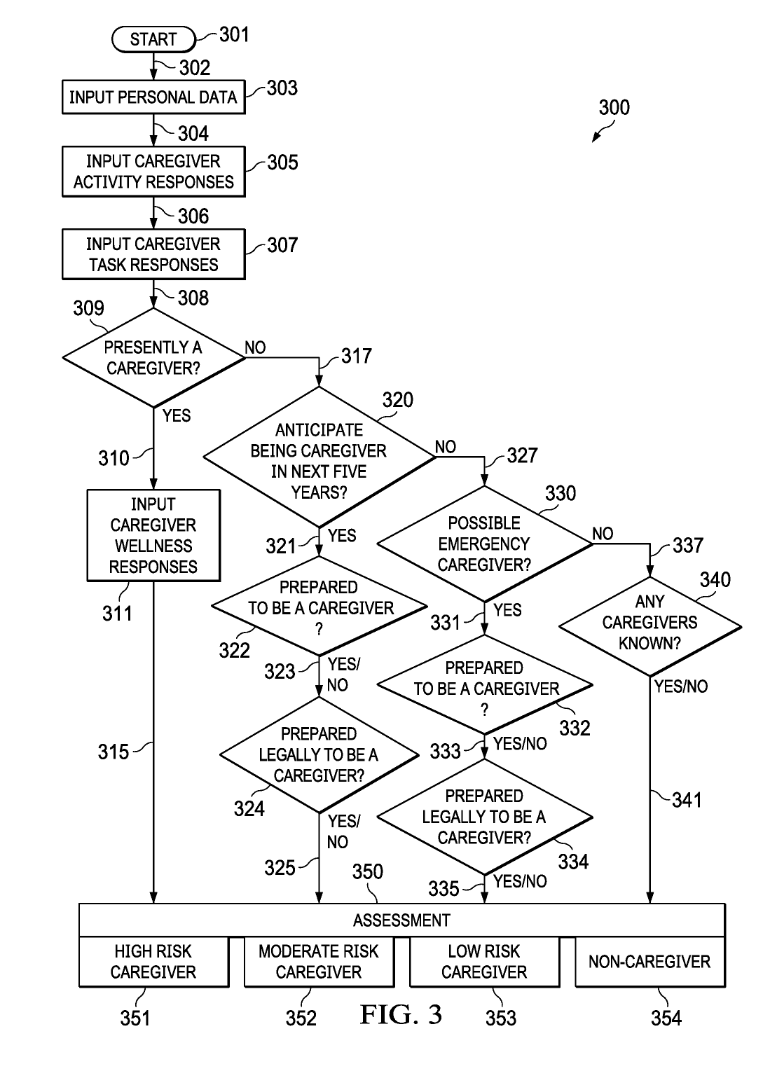

As seen in FIG. 3, an individual user will be walked through a series of decision tree 300 screens asking for information about the assessment taker (Caregiver) and their needs. The assessment survey of the management system and method of the caregiver support platform is a tool that identifies users who need caregiver support and connects them with the resources available to them through the management system of the caregiver support platform.

The assessment sorts users into four categories: caregivers, preppers, perfectly prepared caregivers, and non-caregivers. Upon completion of the assessment, participants are directed to a landing page informing them of the category they fit into and are quickly educated about what it means to be a caregiver, prepper (anticipate being a caregiver), perfectly prepared caregiver (prepared or possible emergency caregiver) or non-caregiver. Caregivers and preppers are also presented with the option by the management system of the caregiver support platform to “Get Started” with the caregiver support platform by setting up a case. This allows them to quickly receive the caregiver support that the assessment has identified them as needing. If the user does not select the “Get Started” option, the user is then automatically added to an email list for the management system of the caregiver support platform that provides further educational content and resources for the user's specific caregiving situation.

The user will access the assessment via the generated caregiver assessment link of the management system of the caregiver support platform. Additionally, the assessment can be taken in multiple languages. The assessment will detect the browser language to determine the best initial language to display.

As seen in FIG. 3, at the start 301 of the assessment, the user is prompted 302 by the management system of the caregiver support platform to input personal data 303, i.e., first and last name, and email address. When the personal data is submitted, the user is directed 304 to input responses to questions about caregiver activities 305 about helping a loved one.

Caregiver Activities:

The Caregiver Activities 305 screen of the management system of the caregiver support platform questions the User about whether they are currently helping a loved one (e.g., parent, spouse, child, differently-abled adult child, in-law, grandparent, etc.) with activities such as, understanding how to pay for care (e.g., reviewing Medicare, Medicaid, Long-Term Care polices, etc.); finding doctors or specialists (e.g., neurologists, cardiologists, oncologists, etc.); filing out legal documents (e.g., Powers of Attorney, Advanced Directives, Living will, etc.); interviewing in-home caregivers; researching or visiting care facilities (assisted living, skilled nursing, memory care, etc.); or none of the above. After clicking OK, all Users are then directed 306 by the management system of the caregiver support platform to the Caregiver Tasks 307 screen.

Caregiver Tasks:

The Caregiver Task 307 screen of the management system of the caregiver support platform questions the User about caregiver tasks including: providing personal care (e.g., bathing, dressing, toileting, grooming, oral care, etc.), meal preparation and/or eating (feeding); managing medication; arranging or providing transportation; assisting with sitting or standing; helping with household chores (e.g., laundry, shopping, cleaning, yardwork, etc.); and none of the above. After clicking OK 308, users are assessed by the management system of the caregiver support platform as to whether they are presently caregivers 309 or not. If the user is assessed by the management system of the caregiver support platform as presently a caregiver, the user is sent 310 to the Caregiver Wellness 311 screens. If the user is assessed as not presently being a caregiver, the User is sent 317 to the first Caregiver Prep screen 320.

Caregiver Wellness:

The Caregiver Wellness 310 screens of the management system of the caregiver support platform question the caregiver about emotional, financial and social wellness. The User is questioned about how caregiving is impacting their time by asking how often in a week the User worries about the loved-one. The user checks a response of never, one or twice, several times, or daily. The User is questioned about how caregiving is impacting them financially by asking if they are currently helping to pay for the care of a loved one (e.g., rent groceries, hospital bills, in-home care services, rehab/therapy, etc.). Yes or no answers are supplied by check-boxes. The user is also questioned about how caregiving is impacting their quality of life by asking if taking care of a loved one has caused them to spend less time at work or with family and friends. Yes or no answers are supplied by check-boxes. After completing the Caregiver Wellness questions, the User clicks to submit 315 the assessment survey, then the responses are analyzed by the management system of the caregiver support platform and a risk assessment 350 is determined.

Caregiver Prep:

If the User answered “none of the above” on the Caregiver Tasks 307 screen, the User is sent 317 to the first Caregiver Prep 320 screen by the management system of the caregiver support platform and questioned (Yes/No) about whether they anticipate needing to provide care for a loved in the next five years. If Yes is checked, the User is sent 321 to the second Caregiver Prep 322 screen. If the answer is No, the User is sent 327 to the first Future Caregiver 330 screen.

The second Caregiver Prep (322) screen of the management system of the caregiver support platform asks the User about how prepared they are to manage a loved one's health emergency. Responses include: I wouldn't know where to start, underprepared, and adequately prepared. The User is then sent 323 to a third Caregiver Prep 324 screen (Prepared Legally) and questioned (Yes/No) whether the User has legal documents, such as Medical Power of Attorney and Living will, for a loved one for whom they anticipate caregiving. After completing the Caregiver Prep questions, the User clicks to submit 325 the assessment survey to the management system of the caregiver support platform, then the assessment responses are analyzed by the management system of the caregiver support platform and a risk assessment 350 is determined.

Future or Possible Emergency Caregiver:

If the User answered No on the first Caregiver Prep 320 screen, the User is sent 327 by the management system of the caregiver support platform to a first Future (possible emergency) Caregiver 330 screen and asked if they have a loved one for whom they would coordinate care in a health emergency (Yes/No). If the answer is Yes, the User is then sent 331 to the a Caregiver Prep screen 332 and questioned about how prepared they are to be a caregiver (wouldn't know where to start, underprepared, and adequately prepared), then sent 333 to the another Caregiver Prep (Prepared Legally) 334 screen and questioned (Yes/No) about whether they have legal documents, such as Medical Power of Attorney and Living will, for their loved one. After completing Caregiver Prep questions, the User clicks to submit 335 the assessment survey, and a risk assessment 350 is determined by the management system of the caregiver support platform.

Future Caregiver:

If the User answered No on the Future (possible emergency) Caregiver Prep 330 question, the User is sent 337 by the management system of the caregiver support platform to a second Future Caregiver 340 screen. The User is asked if they have family or friends currently caring for a loved one (Yes/No). After completing this Future Caregiver screen question, the User clicks to submit 341 the assessment survey, then the responses are analyzed and a risk assessment 350 is determined by the management system of the caregiver support platform.

Caregiver Risk Assessment Results

Result 1: You're a Saint! Positive responses to the Caregiver Activities and Caregiver tasks questions will yield a High-Risk Caregiver 351 assessment from the management system of the caregiver support platform. Falling on the “You're a Saint!” landing page means the user is presently a caregiver. Based on the user's answers to the survey questions, the user has been identified by the management system of the caregiver support platform as providing high-level or hands-on caregiving support to a loved one. The ‘Get Started’ button will allow the user to create an account for the user and will start preparing the care case profile in the management system of the caregiver support platform.

Result 2: You can prepare to care! Negative responses on the Caregiver Activities and Caregiver tasks questions with positive responses on the Caregiver Prep screens will yield a Moderate-Risk Prepper 352 assessment by the management system of the caregiver support platform for a user who anticipates being a caregiver. Falling on the “You can prepare to care!” landing page means the user is a prepper, but may not be fully prepared. Based on the user's answers to the survey questions, the user has been identified as needing to get prepared to be a caregiver. While the user is not currently performing tasks that would qualify him or her as a caregiver, the assessment by the management system of the caregiver support platform identified the user as having loved ones who the user would be responsible for if a medical emergency were to happen or as one who anticipates being a caregiver. The assessment concluded the user would not be prepared in the event of a loved one's medical emergency, making the user fall in the prepper category. Preppers are presented with the option to get started so they can take the necessary steps to get prepared to be a caregiver. The ‘Get Started’ button will allow the user to create an account for the user and will start preparing the care case profile in the management system of the caregiver support platform.

Result 3: You're very prepared! Negative responses on the Caregiver Activities, Caregiver tasks, and positive responses to most Caregiver Prep questions will yield a Low-Risk 353 assessment by the management system of the caregiver support platform. Falling on the “You're very prepared!” landing page means the user is a perfectly prepared caregiver. Based on the user's answers to the survey questions, the user has been identified as being properly prepared to be a caregiver. This means the user has living wills and powers of attorney for his or her loved ones documented and readily accessible. Perfectly prepared caregivers are presented with the option by the management system of the caregiver support platform to share the assessment with others who might not realize they are a caregiver and could benefit from support.

Result 4: You can kick back and relax! Negative responses on the Caregiver Activities, Caregiver tasks, Caregiver Prep and Future Caregiver questions will yield a Non-Caregiver 354 assessment by the management system of the caregiver support platform. Falling on the “You can kick back and relax!” landing page means the user is not a caregiver at the time of the assessment. Based on the user's answers to the survey questions, the assessment identified the user as not having anyone he or she would be immediately responsible for taking care of in the event of a loved one's medical emergency. Non-caregivers are presented with the option by the management system of the caregiver support platform to share the assessment with others who might not realize they are a caregiver and could benefit from support.

Caregiver Assessment for Customer Organization Users

Running the risk assessment survey for customer organizations is particularly beneficial, as a report is generated by the management system of the caregiver support platform for each organization outlining the overall results of the assessment. The assessment report helps organizations understand the overall level of caregiver risk within their organization-employees categorized as caregivers are considered to be elevated-risk, preppers are moderate-risk, and perfectly prepared caregivers and non-caregivers are low-risk.

In order to provide the best possible assistance and utilization of the of the management system of the caregiver support platform, good communication with the Organization is important. The Organization will communicate the list of available employees through an eligibility feed. This provides a list of employees that are eligible for the Caregiver Support service. If a user is already using the service they are converted by the management system of the caregiver support platform from an eligible member to an active member.

The next step is to engage the eligible employees. The following methods can be used: 1) Marketing/Print—The user may receive a printed flyer with the URL on it and a QR code that can be used to convert paper engagement to digital engagement to begin the assessment. 2) Email/Email campaign—The assessment link can be put into a singular email or email campaign. The user will receive the email to click the link to take the assessment. The email campaign can be generated from the organization eligible employee data feed and/or on site capture of emails. 3) Social Media—The assessment link is crafted to be accessible to the social graph allowing it to be sent to target audiences on any available social media platform. 4) SMS—The assessment link can be used to be transmitted via an SMS (short message service) campaign. The link can be shorted via URL shortening service. 5) Link—An assessment link can be sent on any other media that allows the transmission and use of a URL link to allow the end user to run the caregiver assessment by the management system of the caregiver support platform.

The eligibility feed has the ability for the Organization to, also, communicate that they have an employee that is going through a life event. This can be for any reason, such as the Employee is having a baby, the Employee is utilizing Family Medical Leave Act (FMLA) to take time off from work, or any employee for whom the Organization believes the caregiver support service can be of value. The Organization can, also, communicate the event through the Caregiver Support Organization Portal of the management system of the caregiver support platform. The HR or other authorized person can log into the Organization Portal, search for the employee by name, member ID, organization ID, email (work) and email (personal). Once the employee is found, the user can click the Details button to see the Employee details and add the Care Event to the Employee account profile on the management system of the caregiver support platform.

When the information of the Care Event is communicated to the management system of the caregiver support platform it triggers a series of operations. If the Employee already exists as an active member in the management system of the caregiver support platform, the system will find the user account, generate a new case, auto-assign their current support coach and generate a Discussion post. The Discussion post (new post notification) will generate an email to the user's primary email to let them know that the caregiver support service is here to help and the support coach is waiting if they desire the assistance. If the Employee is only an eligible member, the management system of the caregiver support platform will first create the account and then follow the described account process.

In the event of an Employee Care Event, the employer organization can import feed about the event to the management system of the caregiver support platform leading to an account creation. After the account is created a case is created and a support coach is auto-assigned. Case creation leads to generation of a customized email “Can we help?’ post to the employee with an option to click to respond. The employee is then contacted via a message from the management system of the caregiver support platform or by a telephone call from the assigned support coach.

Based on assessment results by the management system of the caregiver support platform from the employee population within an organization, the support platform provider customizes an engagement strategy to fit the specific caregiving needs of employees in the organization. Access of the risk assessment survey through an organization-provided link of the management system of the caregiver support platform identifies the user as an employee of that organization for future account and case creation.

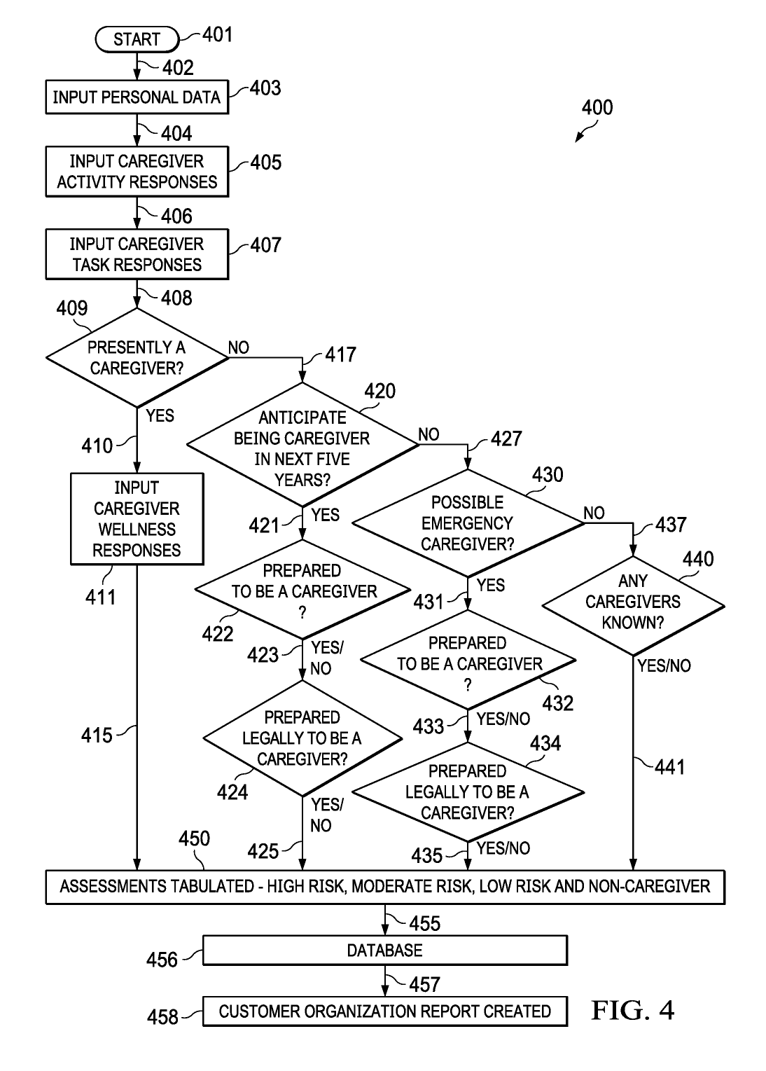

As seen in FIG. 4, an eligible employee user will be walked through a series of decision tree 400 screens by the management system of the caregiver support platform asking for information about the assessment taker (Caregiver) and their needs in the same manner as an individual user, however, prior to the option to create an account, a Caregiver Risk Assessment Report is prepared for the Customer organization.

At the start 401 of the assessment, the user is prompted 402 to input personal data 403, i.e., first and last name, and email address. When the personal data is submitted, the user is directed 404 by the management system of the caregiver support platform to input responses to questions about caregiver activities 405 about helping a loved one.

Caregiver Activities:

The Caregiver Activities 405 screen f the management system of the caregiver support platform questions the User about whether they are currently helping a loved one (e.g., parent, spouse, child, differently-abled adult child, in-law, grandparent, etc.) with activities such as, understanding how to pay for care (e.g., reviewing Medicare, Medicaid, Long-Term Care polices, etc.); finding doctors or specialists (e.g., neurologists, cardiologists, oncologists, etc.); filing out legal documents (e.g., Powers of Attorney, Advanced Directives, Living will, etc.); interviewing in-home caregivers; researching or visiting care facilities (assisted living, skilled nursing, memory care, etc.); or none of the above. After clicking OK, all Users are then directed 406 by the management system of the caregiver support platform to the Caregiver Tasks 407 screen.

Caregiver Tasks:

The Caregiver Task 407 screen of the management system of the caregiver support platform questions the User about caregiver tasks including: providing personal care (e.g., bathing, dressing, toileting, grooming, oral care, etc.), meal preparation and/or eating (feeding); managing medication; arranging or providing transportation, assisting with sitting or standing; helping with household chores (e.g., laundry, shopping, cleaning, yardwork, etc.); and none of the above. After clicking OK 408, users are assessed by the management system of the caregiver support platform as to whether they are presently caregivers 409 or not. If the user is assessed as presently a caregiver, the user is sent 410 to the Caregiver Wellness 411 screens. If the user is assessed by the management system of the caregiver support platform as not presently being a caregiver, the User is sent 417 to the first Caregiver Prep screen 420.

Caregiver Wellness:

The Caregiver Wellness 410 screens of the management system of the caregiver support platform question the caregiver about emotional, financial and social wellness. The User is questioned about how caregiving is impacting their time by asking how often in a week the User worries about the loved-one. The user checks a response of never, one or twice, several times, or daily. The User is questioned about how caregiving is impacting them financially by asking if they are currently helping to pay for the care of a loved one (e.g., rent groceries, hospital bills, in-home care services, rehab/therapy, etc.). Yes or no answers are supplied by check-boxes. The user is also questioned about how caregiving is impacting their quality of life by asking if taking care of a loved one has caused them to spend less time at work or with family and friends. Yes or no answers are supplied by check-boxes. After completing the Caregiver Wellness questions, the User clicks to submit 415 the assessment survey, then assessment is analyzed by the management system of the caregiver support platform and a risk assessment 450 is determined.

Caregiver Prep:

If the User answered “none of the above” on the Caregiver Tasks 407 screen of the management system of the caregiver support platform, the User is sent 417 to the first Caregiver Prep 420 screen and questioned (Yes/No) about whether they anticipate needing to provide care for a loved in the next five years. If Yes is checked, the User is sent 421 to the second Caregiver Prep 422 screen of the management system of the caregiver support platform. If the answer is No, the User is sent 427 to the first Future Caregiver 430 screen.

The second Caregiver Prep (422) screen of the management system of the caregiver support platform asks the User how prepared they are to manage a loved one's health emergency. Responses include: I wouldn't know where to start, underprepared, and adequately prepared. The User is then sent 423 to a third Caregiver Prep 424 screen (Prepared Legally) and questioned (Yes/No) whether the User has legal documents, such as Medical Power of Attorney and Living will, for their loved one. After completing the Caregiver Prep questions, the User clicks to submit 425 the assessment survey, then the assessment responses are analyzed and a risk assessment 450 is determined by the management system of the caregiver support platform.

Future or Possible Emergency Caregiver:

If the User answered No on the first Caregiver Prep 420 screen of the management system of the caregiver support platform, the User is sent 427 to a first Future (possible emergency) Caregiver 430 screen and asked if they have a loved one for whom they would coordinate care in a health emergency (Yes/No). If the answer is Yes, the User is then sent 431 to the a Caregiver Prep screen 432 and questioned about how prepared they are to be a caregiver (wouldn't know where to start, underprepared, and adequately prepared), then sent 433 to the another Caregiver Prep (Prepared Legally) 434 screen and questioned (Yes/No) about whether they have legal documents, such as Medical Power of Attorney and Living will, for their loved one. After completing Caregiver Prep questions, the User clicks to submit 435 the assessment survey, and a risk assessment 450 is determined by the management system of the caregiver support platform.

Future Caregiver:

If the User answered No on the Future (possible emergency) Caregiver Prep 430 question, the User is sent 437 to a second Future Caregiver 440 screen of the management system of the caregiver support platform. The User is asked if they have family or friends currently caring for a loved one (Yes/No). After completing this Future Caregiver screen question, the User clicks to submit 441 the assessment survey, and a risk assessment 450 is determined by the management system of the caregiver support platform.

The possible assessment results by the management system of the caregiver support platform, which indicate the level or likelihood of being a caregiver, are high risk (presently a caregiver), moderate risk (preparing to be a caregiver or anticipate being a caregiver), low risk (fully prepared to be a caregiver) and non-caregiver.

The assessments are tabulated by the management system of the caregiver support platform and risk levels 450 are determined for the employees. The assessments are transferred 455 to the database 456, where the data is analyzed and loaded 457 into a Caregiver Risk Assessment Report 458 deliverable to the Customer Organization. The Caregiver Risk Assessment Report provides the Customer Organization with the following analytical documents: General Assessment Overview, Analysis of High Risk Employee Caregivers, Analysis of Moderate Risk Employee Caregivers, Caregiver Risk Summary, and Next Steps

General Assessment Overview: The overview details the number of employee participants and the Employee Caregiver Risk Levels (high, moderate or low) among the participants. A pie chart is provided showing a breakdown of assessment participants by percentages as presently caregivers, caregiver preppers, perfectly prepared to be caregivers, and non-caregivers.

Analysis of High Risk Employee Caregivers: This analysis provides bar charts showing the most common (high-level) caregiving tasks and the most common (day to day) caregiving tasks of the employee caregivers identified as high risk. Pie charts demonstrate mental wellness indicators, i.e., how frequently the employee caregivers worries about a loved one, financial wellness indicators, i.e., the percentage of employee caregivers who are providing financial support or a loved one's care, and emotional wellness indicators, i.e., the percentage of caregivers who are spending less time at work or with family and friends due to caregiving tasks.

Analysis of Moderate Risk Employee Caregivers: The analysis provides a pie chart demonstrating employee's preparedness to manage a health emergency and legal readiness for caregiving, i.e., having legal documents, such as Medical Powers of Attorney and Living Wills, prepared and accessible.

Caregiver Risk Summary: This summary rates an organization's overall level of caregiver risk. A summary of the high risk and moderate risk employee caregiver analyses is provided along with employee wellness areas impacted.

Next Steps: The Customer Organization is provided with suggestions for opportunities for improvement and engagement opportunities to implement the program. Employees will be given the option of setting up a case with the caregiver support service. The account is maintained and payed for by the employer organization, with the employees providing details for their own care cases in the management system of the caregiver support platform.

Account or Case Creation. The User completion of the assessment by the management system of the caregiver support platform takes information from the assessment answers and uses it to inform next steps. The answers are used to classify the type of caregiver the user is, with the results in Table 1, below.

TABLE 1
Caregiver Assessment Results

| Caregiver Type | Result/Landing Page | Actions |
|---|---|---|
| Caregiver | You're A Saint! | Create Account/case priority 2 |
| Preparer | You Can Prepare To Care! | Create Account/case priority 1 |
| Non-Caregiver | You Can Kick Back And Relax! | Request to share the Assessment link on |
| | | their social network. |
| Perfectly Prepared | You're Very Prepared! | Request to share the Assessment link on |
| | | their social network. |

For assessments with the Non-caregiver and Perfectly Prepared results, the user is encouraged to share the assessment with their social network.

For those assessed by the management system of the caregiver support platform with Caregiver or Preparer results, the user is presented with a ‘Get Started’ button. The ‘Get Started’ button will take the information already submitted and present a screen to assist the user in creating a caregiver support system account and case. The screen displays fields that can be entered including: first and last name; email address; first and last name of the person being cared for; what are the biggest caregiving challenges; if the user is caring for more than one person; and setting and confirming a password.

If the user has provided any of the information for the account creation screen of the management system of the caregiver support platform as part of the assessment, those fields will be removed and the screen will be tailored to only ask for the information that hasn't already been provided. With the information from the assessment, the experience can be personalized with the user's name. The “loved one's” first and last name are used to generate the display name for the case.

Responses to the “Biggest Caregiving Challenge” and the “Are you caring for more than one person?” questions are added to the case notes of the case by the management system of the caregiver support platform. The case notes are used by the support coaches to come up to speed on the case before first contact.

The “Set A Password” and “Password Confirmation” fields are used in conjunction with the email to create an account authentication. The username is the email, and the password is entered by the Caregiver. When the user hits the Submit button, the process of creating an account, creating a care case in the management system of the caregiver support platform, and case startup begins.

The management system of the caregiver support platform also has a system set-up for manual intake of information by a support platform team member. The management system of the caregiver support platform has the ability to define the primary needs that are identified for the case during Intake/Case creation. The Primary Needs section of the manual case creation is data driven by the Primary Needs data defined in the system setup in the management system of the caregiver support platform. Examples of primary needs that may be defined or identified, a few examples of which are: finding facilities, finding doctors, hospital stays, insurance, organic medicines, end of life, legal and financial, public assistance, etc.

The “Add New Case” screen is designed to minimize the time it takes to perform a manual intake/case creation in the management system of the caregiver support platform. During a manual intake, a support platform team member inputs data to fields with the following account owner information at the “profile info” screen: first and last name; primary and secondary emails addresses; phone number; time zone, language preference; case name; case zip code; case priority; coach; and primary needs; how the account owner heard about the service; and additional notes and information. There is a field for organization for cases associated with a customer organization.

For intake of information on a case associated with a customer organization, the intake screen of the management system of the caregiver support platform has a search field is used in conjunction with organization eligibility data feeds to populate the “Add a case” screen fields. The search terms can be any of the following; name, email (primary or secondary) and organization provided Member ID. The “organization” field is a lookup field from the customer relationship management (CRM) list of active organizations with paid contracts. When the organization is selected from the list, the form will auto-reevaluate for case priority and support coach assignment recommendations.

The first and last name fields are the account owners first and last name. The email and secondary email are used to authenticate the user. The two fields are typically used as a way to hold the user's work email and personal email. Both emails are attached to the user account and can be used for authentication and notifications in the management system of the caregiver support platform. The phone number field allows for the user to enter their contact phone number. The phone number can be used for SMS contact.

The time zone and zip code fields let the support coaches know where the account owner or caregiver lives. This information is used to display the account owner's time, weather and other locale related information by the management system of the caregiver support platform. The Case Zip code field is used to identify where the Caregiver lives and the address is updated with the zip code.

The Language preference field is used to set the initial default language that will be used when the user logs into the User Portal of the management system of the caregiver support platform. The Case Name field is the display name of the case. The naming convention used is for the intake person to create a case name based on the loved one's first and last name.

The Case Priority allows the Intake user to define the case priority. While other fields may change this value, the Intake user has the ability to override and define it at the time of case creation in the management system of the caregiver support platform. The Case Priority is related to a coaching skill. This skill (or the ability to handle a specific case priority level) is used to match a case to a support coach.

The Coach field is a calculated lookup field that is updated based upon the organization and the case priority. The initial value of the field is a suggestion by the management system of the caregiver support platform of the support coach to be assigned to the case at case creation. The field can be overridden by the Intake user.

The Primary Needs fields are a set of selections (defined by the set-up of the management system of the caregiver support platform) that, when selected, communicate to the coach, from Intake, what the caregiver's primary needs are. These needs are stored in the coach notes as part of case creation. The needs are also sent to the analytics system of the management system of the caregiver support platform to track primary needs across all cases. The “How did you hear from us?” and “Additional Info” fields are used for analytics tracking by the management system of the caregiver support platform of which assessments and/or media campaigns brought the converted user to the service.

The “Notes” field is a free form field that allows the Intake user to enter any additional notes/information that need to be communicated to the support coach. These notes are added to the case notes at case creation time. When the Intake user hits the Next button, the process of creating an account, creating a care case in the management system of the caregiver support platform, and case startup begins.

Case Creation and Startup

The account is the primary representation of a person in the management system of the caregiver support platform. The account is identified by ID or email. An ID is provided to members who are associated with a customer organization. To create an account, the system uses the information from the assessment and/or manual case creation. The account is created from first name, last name, email, secondary email, password, time zone, phone number and address.

Once an account has been created by the management system of the caregiver support platform, a mirror account object is created in Customer Relationship Management tool (CRM) and a CRM ID is assigned to the account to relate and track the two account objects. For a customer organization, information is used to lookup the organization ID and save it in the Account object to create an Organization to Account relationship. An account represents anyone that uses the caregiver support system. A Care Team member is represented by an Account.

Once the account has been created, a care case is created in the management system of the caregiver support platform. The care case is the primary representation of the interaction between the management system of the caregiver support platform and the Caregiver. The case is created with the information from the assessment and/or manual case creation as follows: Case Name, Case Zip Code and Case Priority. The case is used to track discussions, comments, documents, and care team members.

Coach Assignment.

Once a case has been created by the management system of the caregiver support platform, a support coach is assigned to the case. A support coach may be assigned by a caregiver support team member or assigned automatically by the management system of the caregiver support platform based on case specifics entered by the member. The caregiver support system can define a set of skills and/or capabilities that a coach has to be matched to a case. The skills can be used to match a level of case 1, 2, 3 (low, medium or high case priority); a type of case specialization (for example: pediatrics); or a type of support coach certification that could be required. The case skills are used to match a support coach assigned to a case and/or to drive case setup and system startup behaviors. For example, special Discussion messaging may be auto-generated to pediatrics families.

When a support coach is setup in the management system of the caregiver support platform, the coach's skills and capabilities are entered and attached to the coach's profile. For example, skills and capabilities include proficiency in management of low, medium or high risk cases, pediatric cases, differently-abled adult cases, etc.

The support coach will define a set of information to personalize the coach to the Care Team, such as contact information and a welcome message. The coach will input data in fields with the name to be displayed to the Care Team, (e.g. Coach Fred) along with the email address and phone number the care team can use to contact the coach directly. The Welcome Subject and Welcome message fields are used to create a personalized message from the support coach at the time of Case Setup in the management system of the caregiver support platform. This automated message is an initial Discussion post that the Care Team will see when first accessing the Care Portal.

When an Organization is created in the management system of the caregiver support platform, case options can be defined to influence case support coach assignment, case setup and case startup. This allows for special adjustments to be made by the management system of the caregiver support platform because a case is created in and paid by an organization. For example, when the care case is associated with a customer organization, the case options may state a minimum priority level, special skills (e.g. pediatrics), or other specifics that are set to influence coach assignment to the case.

Once a case is created and paid, the management system of the caregiver support platform will automatically assign a Support coach to the case. The support coach assignment is performed by the management system of the caregiver support platform based upon multiple criteria as defined by the Intake process, such as, Organization Attributes (case Options); Account considerations, Coaching Attributes (skills/capabilities), and Case Attributes (priority, skills/capabilities). As part of Account considerations, if a support coach is already assigned to an existing case, that coach is heavily weighted/considered to be the support coach for the new case for that account, and while being assigned to an existing case is a strong consideration, other factors, such as a support coach's case load, could cause that support coach to be excluded from the new case.

Once all of the criteria has been applied, a list of support coaches becomes available for selection by the management system of the caregiver support platform. From this set of available Support coaches, the management system of the caregiver support platform will look at each support coach's current active case load and any recent prior case assignments. Support coaches having an excessive active case load are removed from consideration. Once a final set of support coaches has been identified, the system will choose the support coach for the case based upon the longest time since they were last assigned a case.

Once the support coach is assigned to a case, a series of automated events occur to setup the case in the management system of the caregiver support platform. The assigned Support coach Welcome message is generated into the case Discussion to introduce the assigned support coach to the Care Team. The caregiver support system has a series of automatic posts by the management system of the caregiver support platform that will be added to the case Discussion. The Discussion posts and Documents that are automatically added can be driven based upon the Skills and Primary Needs noted during account creation. An initial Account setup notification is sent by the caregiver support system to the Account case owner prompting them to setup their authentication credentials. A Welcome email is sent to the Account case owner to start the case engagement. The case is marked as an active case and will be presented at the top of the assigned support coach's Active Cases.

Caregiver Member/User Portal

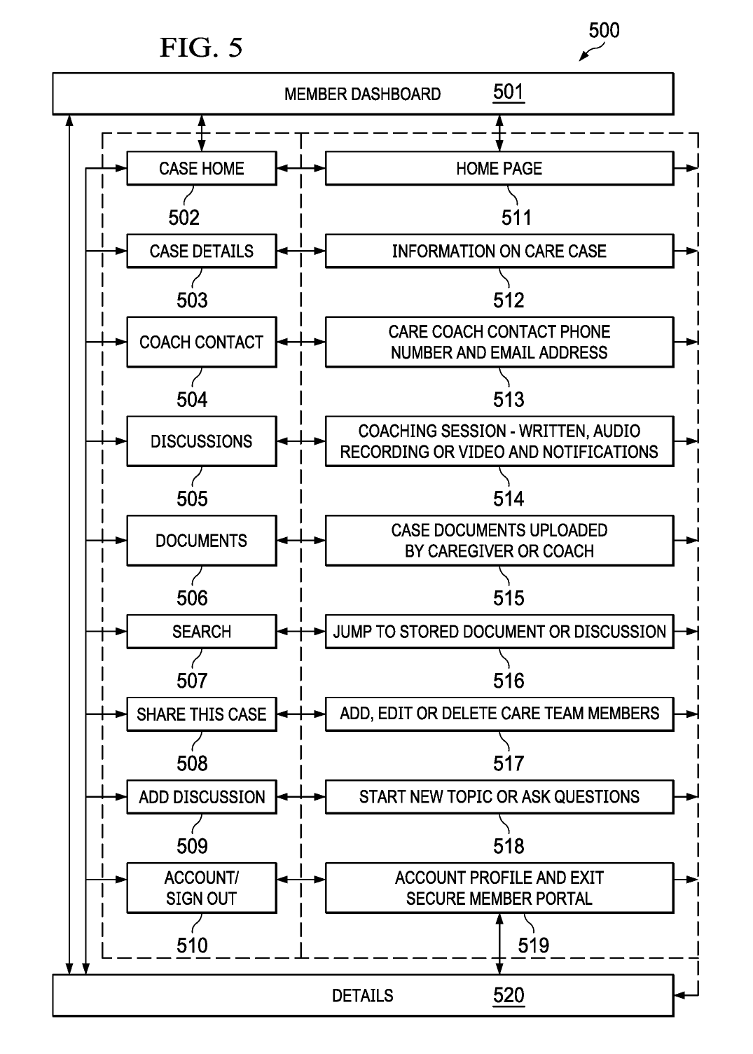

FIG. 5 is an illustrative example 500 of one embodiment of the interaction between the caregiver dashboard and tabs in the management system of the caregiver support platform. Embodiments with dashboards having differing selectable elements are envisioned within the invention to accommodate a user's needs. The caregiver member dashboard 501 will display a plurality of selectable elements specific to a caregiver's established right by the management system of the caregiver support platform s. The caregiver has rights to the care case or cases that are associated with the caregiver account. Tabs are the selectable elements on the dashboard that direct the member to topic specific landing pages. The Case Home tab 502 returns the member to the Main Screen (or Home Page) 511 for the care case. The Case Details tab 504 directs to the details page 512 with information about the care case and care recipient. The Coach Contact tab 504 directs to the telephone number and email address for the support coach assigned to the case. The tab can be personalized with an image or photo of the support coach.

The Discussions tab 505 directs to discussions 514 for the care case by the management system of the caregiver support platform. Discussions 514 includes interactions between the support coach and the care team including comments made by care team member for the discussions, discussions are also stored and will maintain care team comments, text files of written discussions, and audio or video files of recorded coaching sessions. The most recent discussions are displayed on the home page for the care case. Within discussions, caregivers and support coaches can comment on the discussion or post documents related to the discussion. Automated system notifications from the management system of the caregiver support platform are also stored with discussions and the most recent notifications are displayed on the home page.

The Documents tab 503 directs to uploaded documents 515 in the management system of the caregiver support platform that are part of the care case. Documents can be uploaded and stored by care team members or the support coach. Document examples are powers of attorney, living wills, advanced directives, and insurance paper, etc. Most document types can be uploaded to Documents, such as, text documents, PDFs, images, etc. Documents can be uploaded directly or as part of a discussion. Documents that are uploaded as part of a discussion can also be accessed from the stored discussion.

Documents 515 acts as a type of firebox on the management system of the caregiver support platform that stores a set of core documentation that caregivers should have in place to provide the best care for their loved ones. Support coaches educate members on what those documents are, and guides them through finding them, filling out necessary forms and storing the documents securely on the portal where they can be accessed on both desktop and mobile devices at the moment the documents are needed. The Search tab 507 allows the member to locate specific documents or discussions by typing the desired text in the search box. Documents and discussions matching the search criteria will be displayed.

The Care Team (or Share this case) tab 508 of the management system of the caregiver support platform directs to an option 517 to share the case with other caregivers, such as family and friends who assist in care giving, or if care team members have been authorized by the primary caregiver/account owner, a list of care team member is displayed. Care team members can be added, edited or deleted.

The Add Discussion tab 509 of the management system of the caregiver support platform opens a new discussion 518 field where the member can ask questions or start a new topic of discussion for the support coach and/or other care team members. Care team members can comment on the new discussion or upload documents related to the new discussion. New discussions and related comments are displayed on the main page and will also be maintained with the stored discussions 514.

The Account tab 510 of the management system of the caregiver support platform directs to a dropdown menu 519 with the account profile and sign out as options. The account profile has details about the caregiver account entered at account creation, and the user can edit the account profile details. The account profile also has account status details and payment management details. The account tab can be customized to greet the member by name. The sign out tab exits the secure portal.

The member dashboard illustrated in FIG. 5 is a representation of the types of dashboards that can be used in the management system of the caregiver support platform. Selectable items on the dashboards represent types of tasks that may be performed by the dashboard user. Tasks and selectable items may be added or deleted depending on the display rights and/or needs of the user

Caregiver members are able to access the Care Portal of the management system of the caregiver support platform through the Personal Portal Login Screen. At login, the user can select the preferred language for interaction. The user's email and password are used for authentication by the management system of the caregiver support platform. A “forgot password” link is provided to allow the user to reset the password for the email/account. The Care Portal also allows the user the option to authenticate using federated social media. Once authenticated the user is allowed access to the Care Portal which is shared by all members of the Care Team including the assigned support coach.

Once the member's login has been validated by the management system of the caregiver support platform, the member is taken to the account home page which displays the member dashboard. The page displays the Case Selector, which will display the last accessed and current Care Case. If the caregiver user account has multiple cases, the user will be presented with a list of available cases and the user can select the case to view. A user can be involved as a case owner or can be someone who has been invited to be a member of the Care Team on the case.

A menu on the home page of the management system of the caregiver support platform welcomes the user by saying “hello” and displaying their first name. When selected it will present menu options that are relevant to the account, including an account option and a Sign Out option. Sign Out will de-authorize the session and return the user to the login page.

The Account selection of the management system of the caregiver support platform will present to the user a screen displaying the current details for User Profile with a prompt to keep the profile information updated. The User Profile fields include first and last name; primary and secondary email, phone number, password and password confirmation, preferred language, and cases. The Account screen allows the user to update and personalize the information displayed on the page. The user can update information including: name, contact information, such as email and phone number, and update the password. If an account has lapsed, the user can also renew cases they own by applying payment.

The user can personalize the page by creating an image to be displayed as part of the Discussions and comments, as well as, identify them visually to the other members of the Care Team. The user can select their primary language that drives the display of all screens of the Care Portal on the management system of the caregiver support platform. The user can rename cases they own and join cases they've been invited to join.

Discussions. The account home page of the management system of the caregiver support platform displays recent discussions. The discussions section is the primary interface for user and support coach interactions. Care team members can comment on the discussions or upload documents related to the discussion. Comments and uploaded documents are maintained with the discussion in the care case and will be accessible to the care team members and support coach. The discussions section also displays automated notifications from the management system of the caregiver support platform, including, but not limited to, messages welcoming the user, support coaches assigned or removed from the case, survey requests and options to renew a lapsed account. The discussions can be sorted by their most recent update date of the discussion posts by either the newest or oldest posts for the case.

Care Team members can create a new post by clicking the Add Discussion button on the management system of the caregiver support platform. When the button is pressed the user is presented with an inline form that allows them to enter the title/subject of the discussion. Additionally, the user can enter in a rich text formatted body/message that communicates to the entire Care Team, including the support coach. The user can also attach any documents that will be made accessible to the Care Team. Care team members can comment on the discussions or upload documents related to the discussion. Comments and uploaded documents are maintained with the discussion in the care case and will be accessible to the care team members and support coach. Document types including, but not limited to, PDFs, text documents, spreadsheets, slide shows, images, or other documents that communicate information to the Care Team can be uploaded to the case and stored in the management system of the caregiver support platform. The page also has a Cancel button should the user decide not to create a discussion.

Discussion posts on the management system of the caregiver support platform are created by a member of the Care Team (either Support Coach or Care Team Member). A discussion post is composed of several elements, including the name and an image of the Care Team Member making the post, and the date and time the discussion post was made or updated. The next section is the subject and body/message of the post. The edit section allows the creator of the post the ability to edit or delete the post. An example of a Coach started discussion would be a post to Care Team members about documents that are needed in which the support coach is asking for the user to create a post with an attached document. A Care Team member may respond to the Coach's post by uploading the requested documents. The Care Team member post has the same sections (name, image, edit, etc.) as defined by the support coach's post. Members of the Care Team can click on the document icon, and be taken to a Discussion Post Detail screen, and be allowed to see the titles of the uploaded documents. Documents can be downloaded from the management system of the caregiver support platform and/or viewed the documents in the web browser.

Discussion posts may also be inline system messages. The messages may be Coaching changes (assignment, removal), Care Team changes (Invitation, acceptance, removal), and notifications about the activation of the case indicating a support coach has been assigned by the management system of the caregiver support platform and is available to the Care Team.

The Caregiver system notifications by the management system of the caregiver support platform allows the system to communicate messages to the Care Team through the Care Portal about the account. These messages are “sticky” in nature in that they will always remain at the top of the Discussions screen. These type of messages are generated by the management system of the caregiver support platform and can be used to communicate changes to the case as performed by the management system of the caregiver support platform (CSG). For example, these messages may be membership or payment notifications about a case that is expiring, has expired, requires payment, or has been renewed.

A discussion post about the account is generated by the management system of the caregiver support platform when the case has expired to indicate to the Care Team there has been a significant change in the case. If the case has expired, the Care Team is notified and informed that the support coach has been removed from the Case.

A survey-type discussion post is automatically generated by the management system of the caregiver support platform based upon a defined number of interactions by the support coach, by the Care Team or by both the Care Team and support coach. When the threshold of interactions is reached, the management system of the caregiver support platform will automatically generate the custom survey discussion post to encourage all members of the Care Team to provide feedback.

The Care Portal of the management system of the caregiver support platform is designed to support any type of Discussion post that is displayed to the Care Team. The format of the post will always be the user, user name, date and time. The rest of the post is available for any User Interface (UI) components and interactions. The Survey Request is an example of a custom post type that requests the user interaction of Take Survey. The content section can be formatted in any way including text, graphic images, UI elements (such as buttons, sliders, check boxes, selector boxes) and any UI element that is supported by a web browser.

Documents. The Documents section of the management system of the caregiver support platform is a consolidation of all uploaded documents from Discussion posts or directly uploaded to the Case. The Documents section creates a “safe box” for documents allowing the Care Team to know where to find and access the documents. The Documents are accessible from a Computer Desktop or a Mobile Device (e.g., mobile phone, tablet, etc.) Each row of the Documents sections represents an uploaded document and has an icon for the document type (PDF, etc.), a document name, name of the uploader, and time and date of upload. The document can be viewed by clicking the document name. The user has the option to download the document to a local device (laptop, mobile, etc.). The user also has the option to view the discussion where the document was uploaded, allowing the user to see the file upload in context of the discussion post.

Main Navigation. The Main Navigation bar on the dashboard of the management system of the caregiver support platform has a Search field that allows the user to enter text that can be found in a discussion post, discussion comment or file name/description, and will display a list of results that match the defined search criteria. Row of the search results will show a discussion or document that matches the search terms. The user can then click on a discussion to see discussion details or click on the document to download or preview it.

The Main Navigation bar has a Care Team field. The Care Team is the group of people that are involved with the Case. This team consists of a support coach, Care Team owner and Care Team members. The Care Team members can be anyone who the Care Team owner believes should be involved or informed regarding the case and its progress.

The Care Team screen on the management system of the caregiver support platform has a button at the top called Invite Someone. When the user clicks on this button an inline dialog appears allowing the user to invite someone to join a case. The user will enter the invited user's first name, last name and email address. Once the information is entered, the user can click the Send button to send an email invitation to the invited user by the management system of the caregiver support platform. If the invited user already exists as an Account in the system, the case will immediately become available to them in their Care Portal case list.

The Care Team screen of the management system of the caregiver support platform displays the users with name, icon, contact information, their roles and their status on the team. Status can include Coach (user is a support coach), Owner (user is the account owner, primary user responsible for payment and case decision making), and Pending (user has been invited but not accepted yet). There is also a Remove button which will remove a Member's access to a case.

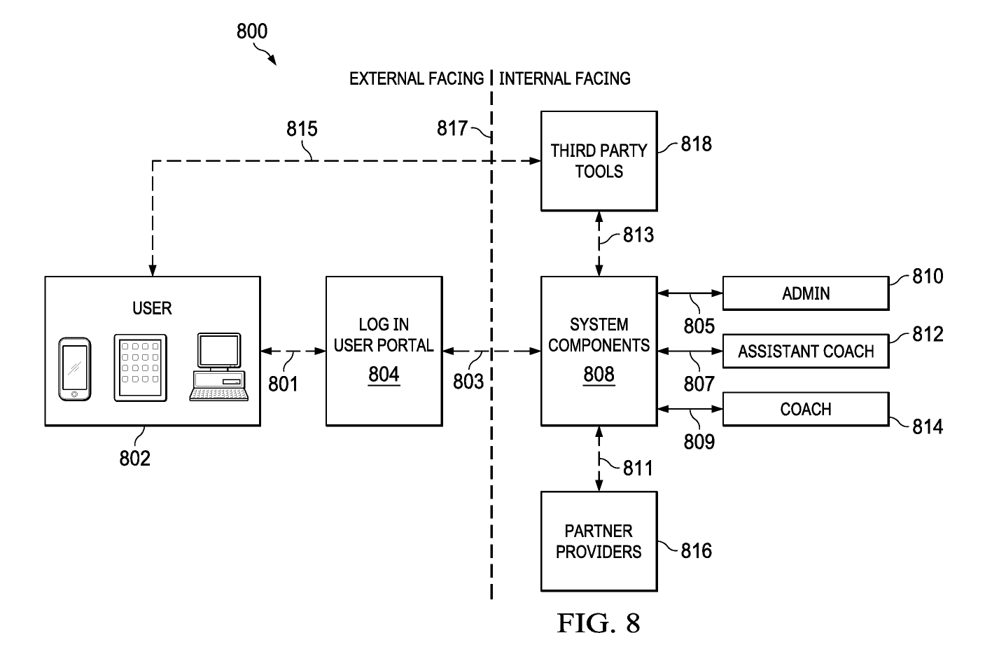

FIG. 8 is an illustration of the interactions between a Caregiver Member and segments of the management system of the caregiver support platform. The management system of the caregiver support platform has external and internal facing portions 817 with the external facing portion facing a member user and the internal facing portion facing the caregiver support platform personnel.

The member user 802 uses a device such as a desktop PC, tablet, mobile phone or other device to access 801 the user portal login 804 of the management system of the caregiver support platform to input email address and password. When the user's credentials are accepted by the management system of the caregiver support platform, the user can access 803 the system components 808 for the management system of the caregiver support platform. Once the user 802 has logged in, the user can view care case documents and discussions, start a new discussion or communicate 807 with a support coach 812 assigned to the case.

The support coach 814 communicates 809 with the user 802 via the management system of the caregiver support platform 808 through discussions on the care case or electronic messaging with the user. Support coaches can access resources such as documents, research and articles in the caregiver support platform knowledge library and make these resources available to a member through a care case discussion. Assistant coaches 812 may also communicate 807 with a user 802. Assistant coaches may help with new case Intake or assist support coaches in the care case. Support coaches 814 may also suggest services with partner providers 816 that members can link to 811 through the management system of the caregiver support platform 808 in order to enroll with a service provider.

Admin 810 can communicate 805 with the user 802 via the management system of the caregiver support platform 808. Admin communication may include account status and payment notifications, survey requests and automatically generated messages from an Artificial Intelligence (AI) coaching assistant. AI coaching assistant messages may include welcome messages, follow-up messages and other messages designed to stay in touch with care team members. Third party communications 818, such as tools for email marketing, may also be directed 813 by the management system of the caregiver support platform 808 to send messages 815 to a user 802. Notifications by an AI coaching assistant may also be automatically generated from the management system of the caregiver support platform 808.

Support Coach Portal.

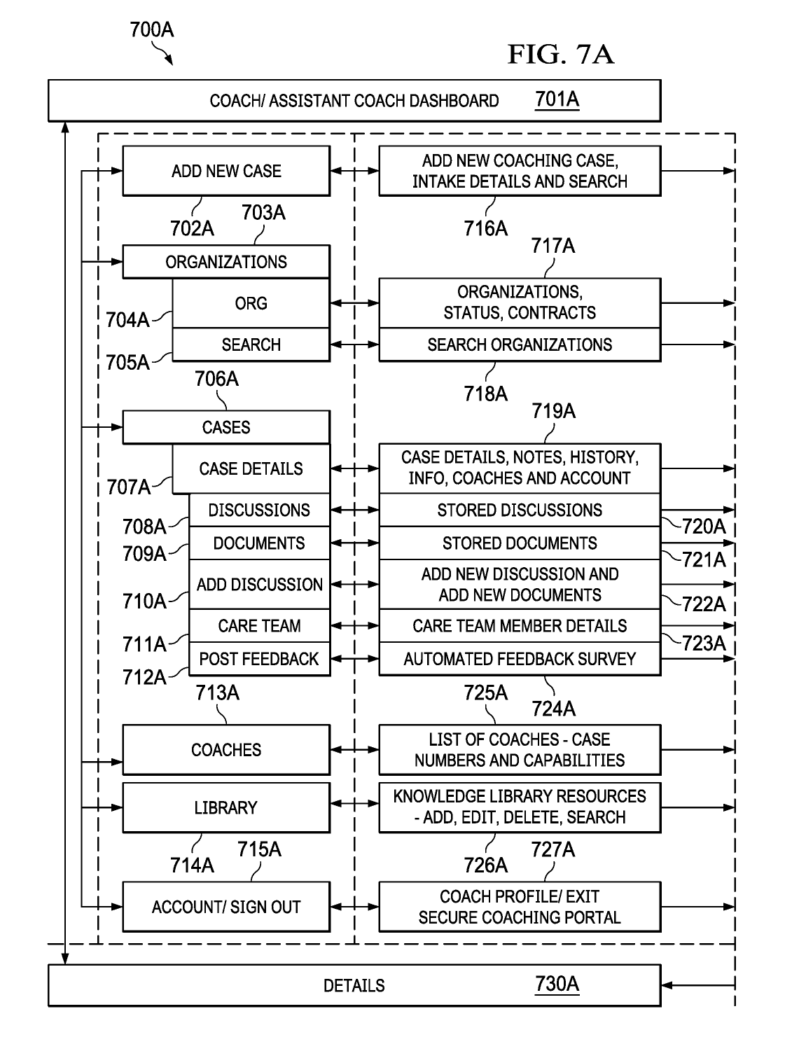

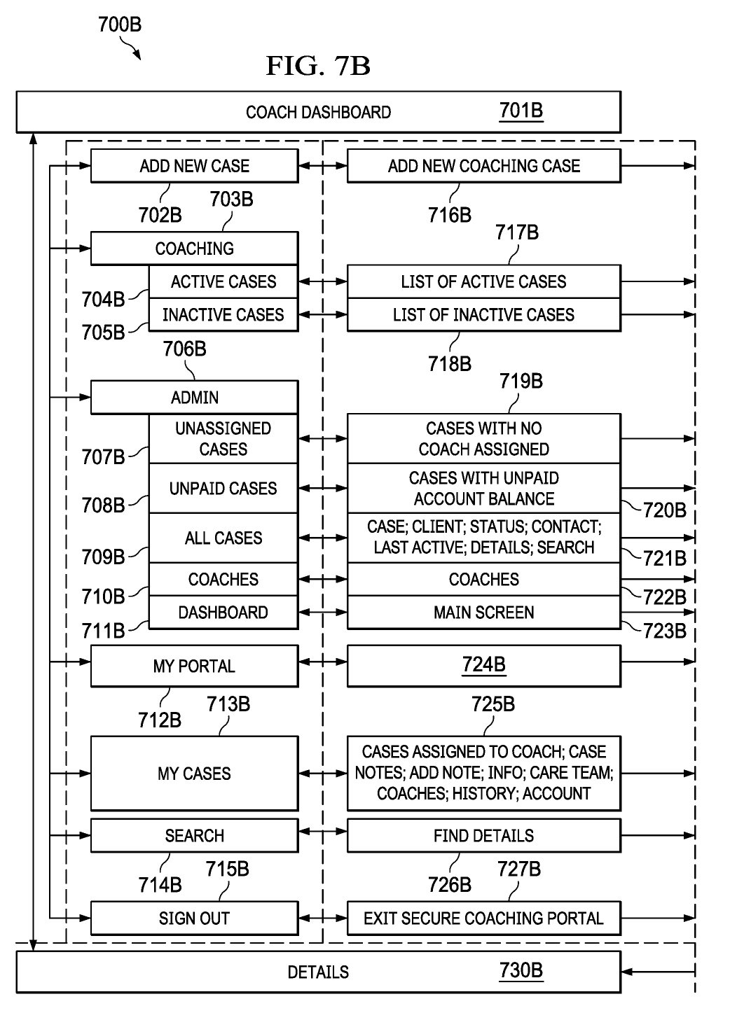

FIG. 7A is an illustrative example 700A of one embodiment of the interaction between the support coach dashboard 701A and tabs by the management system of the caregiver support platform. Embodiments with dashboards having differing selectable elements are envisioned within the invention to accommodate a user's needs. The coach dashboard 701A will display a plurality of selectable elements specific to a support coach's established rights. The support coach has access to the plurality of care cases assigned to the support coach as well as caregiver support platform details, information and resources. Tabs are the selectable elements on the dashboard that direct the coach to topic specific landing pages. The coach home page has a plurality of selectable elements including a search function and tabs of which mirror some of the tabs seen on the member dashboard to facilitate assisting caregivers.

The Add New Case tab 702A of the management system of the caregiver support platform directs to a new case page 716A with an intake form to input new case information and an option to search related cases. The intake form has fields like those on the account profile form for first and last name; primary and secondary emails addresses; phone number; time zone, language preference; case name; case zip code; case priority; coach; and primary needs; how the account owner heard about the service; additional notes and information, and a field for organization for cases associated with a customer organization. The Organization tab 703A directs to a customer organizations page 704A displaying 717A organization name, status and contract, and has an option to search 705A the organizations.

The Cases tab 706A of the management system of the caregiver support platform directs to the support coach's assigned cases 707A with case details 719A including notes, history, info coaches and account profile. Notes are presented in chronological order and are seen on the internal facing portions of the management system of the caregiver support platform and include caregiver challengers noted at account creation, and support coach or assistant coach notes that are not viewable by the member. History includes a running tab of the interactions for the care case on the management system of the caregiver support platform. Everything from manual posts to automatic notifications are captured by the history and can be useful to confirm when actions were taken. Info contains the profile information for the account that was collected as part of the intake process. Coaches includes a list of coaches currently assigned to the care case. Account has information on the account, such as case identifiers, parent organizations associated with the account, and a list of who receives notifications for the account. Tabs on the Case view 707A direct to additional landing pages similar to those seen on the member dashboard, such as discussions 707A, documents 709A, add discussion 710A, and care team 711A. There is also a “post feedback survey” tab 712A.

The Discussions tab 708A of the management system of the caregiver support platform directs to stored discussions 720A for the care case. Stored discussions 720A include interactions between the support coach and the care team, such as text files of written discussions, and audio or video files of recorded coaching sessions. The most recent discussions are displayed on the home page for the care case. Automated system notifications by the management system of the caregiver support platform are also stored with discussions and the most recent notifications are displayed on the home page

The Documents tab 709A of the management system of the caregiver support platform directs to stored uploaded documents 721A that are part of the care case. Documents can be uploaded and stored by care team members or the support coach. Document examples are powers of attorney, living wills, advanced directives, and insurance papers, etc. Most document types can be uploaded to Documents, such as, text documents, PDFs, images, etc. Documents can be uploaded directly or as part of a discussion. Documents that are uploaded as part of a discussion can also be accessed from stored the discussion. The Add Discussion tab 710A opens a new discussion 722A field where the member can ask questions or start a new topic of discussion for the support coach and/or other care team members. New discussions are displayed chronologically on the main page of the management system of the caregiver support platform.

The Care Team tab 711A of the management system of the caregiver support platform directs to a list 723A of care team members who have been authorized by the primary caregiver/account owner. The list indicates the status of the care team members such as account owner, support coach, member, etc. The support coach can assist a caregiver in adding members to the care team.

The Post Feedback Survey tab 712A of the management system of the caregiver support platform directs to feedback surveys 724A. The feedback results are typically auto-posted, however, the support coach has the option to manually post the feedback survey. If the support coach chooses to manually post the survey then the auto-post is canceled.

The Coaches tab 713A by the management system of the caregiver support platform directs to a page 724A with list of caregiver support platform support coaches with information on the number of cases assigned to each support coach, and the capabilities or skill for each support coach.

The Library tab 714A of the management system of the caregiver support platform directs to the caregiver support platform knowledge library 726A with the landing page displaying searchable folders organized by resource type, such as assisted living facilities, hospice information, that a support coach can use to research information specific to the needs of a caregiver. Documents can be added, edited and deleted. There is a search option to find information within the list and within the folders. The support coach also has the option within the library to post documents to a discussion in a care case.

When a member signs up with caregiver support service via the intake team or website the information gathered by the management system of the caregiver support platform is used to determine how severe their needs are and which support coach to assign to the case. The information gathered allows the system to automatically level the case and assign a support coach.

The Coach account tab 715A of the management system of the caregiver support platform directs to a dropdown option 727A to select the support coach's profile information or to sign-out. The support coach can edit the profile or sign-out of the support coach portal. The tab can be customized with the support coach's name.

FIG. 7B is an illustrative example 700B of another embodiment of the interaction between the support (or care) coach dashboard 701B and tabs by the management system of the caregiver support platform. Embodiments with dashboards having differing selectable elements are envisioned within the invention to accommodate a user's needs. The coach dashboard 701B will display a plurality of selectable elements specific to a support coach's established rights. The support coach has access to the plurality of care cases assigned to the support coach as well as caregiver support platform details, information and resources. Tabs are the selectable elements on the dashboard that direct the support coach to topic specific landing pages. The support coach home page has a plurality of selectable elements including a search function and tabs of which mirror some of the tabs seen on the member dashboard to facilitate assisting caregivers.

The Add New Case tab 702B of the management system of the caregiver support platform directs to a new case page 716B with an intake form to input new case information and an option to search related cases. The intake form has fields like those on the account profile form for first and last name; primary and secondary emails addresses; phone number; time zone, language preference; case name; case zip code; case priority; support coach; and primary needs; how the account owner heard about the service; additional notes and information, and a field for organization for cases associated with a customer organization.

The Coaching tab 703B of the management system of the caregiver support platform directs to a pages for active cases 704B and inactive cases 705B. Active cases 704B displays a list of active cases 717B and details for cases that are currently being managed by a support coach in the management system of the caregiver support platform. Active cases may be either individual cases or cases associated with a customer organization. Inactive cases 705B displays a list of inactive cases 718B and details for cases that are no presently being managed by a support coach. Inactive cases includes cases for accounts closed voluntarily or because of lack of payment. Inactive cases also includes cases where the care recipient is deceased.

The Admin tab 706B of the management system of the caregiver support platform directs to administrative details for cases, including unassigned cases 707B, unpaid cases 708B, all cases 709B, coaches 710B and dashboard 711B. Unassigned cases 707B directs to a case listing 719B for cases that do not have a support coach presently assigned. Unpaid cases 708B directs to a case listing 720B for cases with unpaid account balances. All cases 709B directs to a complete case list 721B in the management system for the caregiver support platform, including case details, client information, case status, contact information, and last active date, and includes an option to search within the cases. Coaches 710B directs to a page 722B with list of caregiver support platform coaches with information on the number of cases assigned to each support coach, and the capabilities or skill for each support coach. The Dashboard tab 711B directs back the main screen 723B. The My Portal tab 712B directs to a page 724B with the support coach's profile information and resources. The support coach can add or edit information on the profile.

The My Cases tab 712B directs to the support coach's assigned cases 725B. The case details include case notes with ability to add notes, info, care team members, coaches, history and account. Notes are presented in chronological order and are seen on the internal facing portions of the management system of the caregiver support platform and include caregiver challengers noted at account creation, and support coach or assistant coach notes that are not viewable by the member. History includes a running tab of the interactions for the care case on the management system of the caregiver support platform. Everything from manual posts to automatic notifications are captured by the history and can be useful to confirm when actions were taken. Info contains the profile information for the account that was collected as part of the intake process. Coaches includes a list of support coaches currently assigned to the care case. Account has information on the account, such as case identifiers, parent organizations associated with the account, and a list of who receives notifications for the account.

The Search tab 714B opens a search window 726B where cases can be searched by entering the desired text. The Sign Out tab 714B will exit 727B the coaching portal.

The coach dashboards illustrated in FIGS. 7A and 7B are representations of the types of dashboards that can be used in the management system of the caregiver support platform. Selectable items on the dashboards represent types of tasks that may be performed by the dashboard user. Tasks and selectable items may be added or deleted depending on the display rights and/or needs of the user.

Cases are labelled Level 1, 2 or 3, with higher numbers assigned to cases requiring more attention. Some organization accounts default to a higher level because they are consistently higher maintenance. The levels can be adjusted based by the management system of the caregiver support platform on the amount of attention a case needs by the support coach (not case ailment severity). Support coach interaction is tracked and the levels can be adjusted up or down accordingly. This allows for more accurate determination of a support coach's workload and allows the management system of the caregiver support platform to more accurately auto-assign cases to support coaches based on workload.

Once a support coach has been assigned to a case by the management system of the caregiver support platform, the Coaching Portal of the caregiver support system is where the coaching team (intake, assistant coaches and support coaches) work to bring caregivers into the system and support them.

The Case view screen of the management system of the caregiver support platform is where support coaches interact with members using a view that is very similar to what the member sees for better member support. From this screen the support coach, or other user, will have options to add a discussion, sort discussions, view discussions, add documents, view documents and search, in a manner similar to the portals for the case owner and care team members.

In addition to the features visible in the case owner's view, the coaching portal view of the management system of the caregiver support platform also has a column displaying helpful information, such as case owner's name, contact information, and time zone, next scheduled auto-check-in, date of last discussion, and membership date, as well as actions that the support coach can take, such as posting the feedback survey to discussions. The name and location of the member's loved one is displayed. If the member is associated with a customer organization, the organization name and organization benefits info is displayed with the case data. Support coaches can also assist team members to invite someone to their care team, help a member gain access to the portal if the password is not working, and can resend the password setup email for a member of the care team.

The Coach portal view of the management system of the caregiver support platform has a “Notes View” section. Notes may be entered during the manual intake process by the intake user. The case owner's response to the “biggest caregiving challenge” is also stored as a note. Support coaches and assistant coaches may also add notes that will not be seen by the caregiving members. The support coach or assistant coach can click “notes” to view notes or “add note” to enter a new case note. Notes view will display the notes in chronological order.

The Coach view portal of the management system of the caregiver support platform has a “history view” section. The history is a running tab of the actions that have been taken by the support coach, member and system. Everything from manual posts to automatic notifications are captured by the History. This history can be referenced by the support coach when troubleshooting as confirmation that expected actions have taken place, including when support coach visits to the case occurred, Admin notifications about the account, and automated notifications. The Coach view portal of the management system of the caregiver support platform also has an “Info view” section containing the information for the account owner collected as a part of the intake process. Coaches have the ability to edit this information as needed.

The Coach view portal of the management system of the caregiver support platform has a “Coach list view” section with a list of support coaches currently assigned to that case. The Coach view displays the names, number of assigned cases and capabilities for each support coach on the list. The “coach list view” also has the option to add or remove support coaches from the case.

The Coach view portal of the management system of the caregiver support platform has an “Account Info view” section which communicates account specific details, such as Case identifiers, links the customer relationship management (CRM) tools, ability to re-parent the case, and a list of those receiving notifications and the ability to disable those notifications.

The Coach view portal of the management system of the caregiver support platform has an “Intake view” section where the support coach can add a case. The support coach can view the intake form which the intake team fills out when onboarding new members. This information is what also individual users fill out the Profile info when they create a case directly from the website and/or assessment by the management system of the caregiver support platform, as well. The fields for the intake form include: Name and organization info; Contact info; Language preference; Case details (location, severity, coach); Primary Needs; Awareness tracking (how did you hear about us?); and Notes (these get saved to the case's notes section to be accessed by the support coach once case is created). For some customer organizations, there is an eligibility file connected to the caregiver support system which helps with understanding an employee's eligibility and can populate certain data fields.

The Coach view portal of the management system of the caregiver support platform has an “Organization List view” section where the support coach can see what organizations are currently offering the caregiver support platform to their employees, as well as the status and contract type for the organization.

Knowledge Library. Support coaches also have access to a knowledge library hosted by the management system of the caregiver support platform. Caregiver support members including admin, assistant coaches and support coaches Admin also have access to the knowledge library. The knowledge library is hosted on the management system of the caregiver support platform for easier/better file management, so the support coaches are able to easily share documents with caregiving members via the portal. The information shared can also be tracked to uncover trends.

Support coaches can access the knowledge library on the management system of the caregiver support platform by clicking on the library button on the Coach view portal screen. Folder rows are displayed with descriptive titles (e.g., affordable housing, articles, assisted living, etc.). Users single click a row to highlight and reveal a menu button. Right-clicking the row or clicking on the menu item reveals the option menu. Double-clicking a folder row will take the user into that folder. The Option Menu is revealed when a user right-clicks on a row or clicks the menu button that is revealed when a row is selected. Clicking on the row opens the folder.

Several options for actions for folders are available. Clicking “Move” allows a file or folder to be moved. Clicking “Rename” opens the rename modal. Clicking “Delete” launches a confirmation alert prior to deleting the item. Each folder also displays “Last Modified” indicating the last time a folder had new content added or updated. Individual files also have descriptive tags displayed. When a folder is opened, a list of individual files appears that are related to the folder title. For example, if an “Assisted Living” folder is opened, all of the files in the folder are documents related to assisted living information, facilities, contacts, etc.

Individual file rows in each folder follow the same row highlight/interactions as stated on the Folder Row page. Breadcrumb navigation is available when accessing files in the folders. Hovering over “Add New” will reveal the Add New options. Clicking Document navigates to a screen to add a new document. Clicking Folder opens a modal to enter a folder name. Right clicking a row or clicking on the menu of a row reveals the option menu for files. If the file has a short description it can be added to the top of the menu.

Several options for actions are available for files in the folders of the management system of the caregiver support platform. Clicking preview (or double-click a file row) opens a preview in a new browser tab. Clicking edits opens the edit screen. Clicking Download initiates a download. Clicking Move follows the same pattern as “move” on the Categories screen. Clicking Send opens a modal to accept an email address to send the file to a member with the file name as the subject of the email. Delete has the same function as stated on the Folder Row bullets. Files display tags for quick reference for support coaches providing information about the document to determine if it's the one need. Tags are specific to files, not folders. For files, Last Modified is the last time the file was posted or replaced.

The knowledge library of the management system of the caregiver support platform can be searched by entering the desired text in the search field to display a list of matches showing the title or tag associated with the search text. Users can click on an item in the list to view the document or click on Back to navigate back to the previous screen prior to the search.

New folders can be added by clicking “Add Folder.” Naming the folder and hitting save will save the new folder to the knowledge library. Clicking cancel will remove the new folder. Existing folders can be renamed by clicking the rename modal, entering the new name and hitting save.

New documents can be added to the knowledge library of the management system of the caregiver support platform. A file is chosen from the computer to add to library. The file name is loaded into the field so it is can be edited, if needed, editable before posting. Descriptive tags can added to the files. A file Description is optional. If entered, it is displayed it at the top of the menu options. New file can be stored in a folder or added directly to the library of the management system of the caregiver support platform. File addition can also be canceled by clicking on “Cancel.” Documents in the knowledge library can be replaced and descriptors for documents can be edited. Click Replace to upload a replacement file. The existing name and tag and descriptions can be edited. A User can edit an existing description or add one if there was not one before. Clicking Cancel will send the User to the previous screen.

Coaches have the ability to grab a file from the knowledge library of the management system of the caregiver support platform and post to a discussion while they are creating a new discussion or adding a comment to a discussion. The support coach's dialog box for a discussion has a “Knowledge Library” button that allows the support coach to access the library and post a document to the discussion.

The Knowledge Library button in the dialog box will open a finder window with a search box. The support coach can search based on the desired terms. Documents matching the search terms are displayed in the finder window. When a file is selected the details are displayed in the file detail area. A Preview can also be reviewed by the support coach before posting it to make sure it's the right one. Clicking “preview” will open the file in a new browser tab. When ready to attach a file, the support coach will click “attach.” Files can be attached, but folders cannot. If a folder is selected, clicking X or Cancel will close the finder window.

Caregiver Services. Caregiver Services are third party functionalities that are made available to caregiving members via the management system of the caregiver support platform portal. The management system of the caregiver support platform ties into the third party APIs providing an interaction layer within the caregiver support system portal so members can take advantage of the specific services while logged into the portal of the management system of the caregiver support platform.

A number of services associated with Preferred Providers are made available to the member. Members may have access to all Services or only a subset depending on membership type, need, etc. Users can filter by service type and click on a service button to learn more, activate or sign up for that service. Service categories may include transportation services, food services, legal services, home care services, personal care services, medication services, product delivery services, etc. and other services that would assist a member in caring for a loved one's needs.

Members can access information on available services through the management system of the caregiver support platform and view details about the services in a category or about a specific service. Each service has a detail page that provides information about that service as well as a method for activating that service. From the management system of the caregiver support platform portal, members can view the detail page for a service, view the unique presentation for using that service (e.g., view a prescription card), and link to the selected service sites to activate or sign-up for that service.

Admin Portal

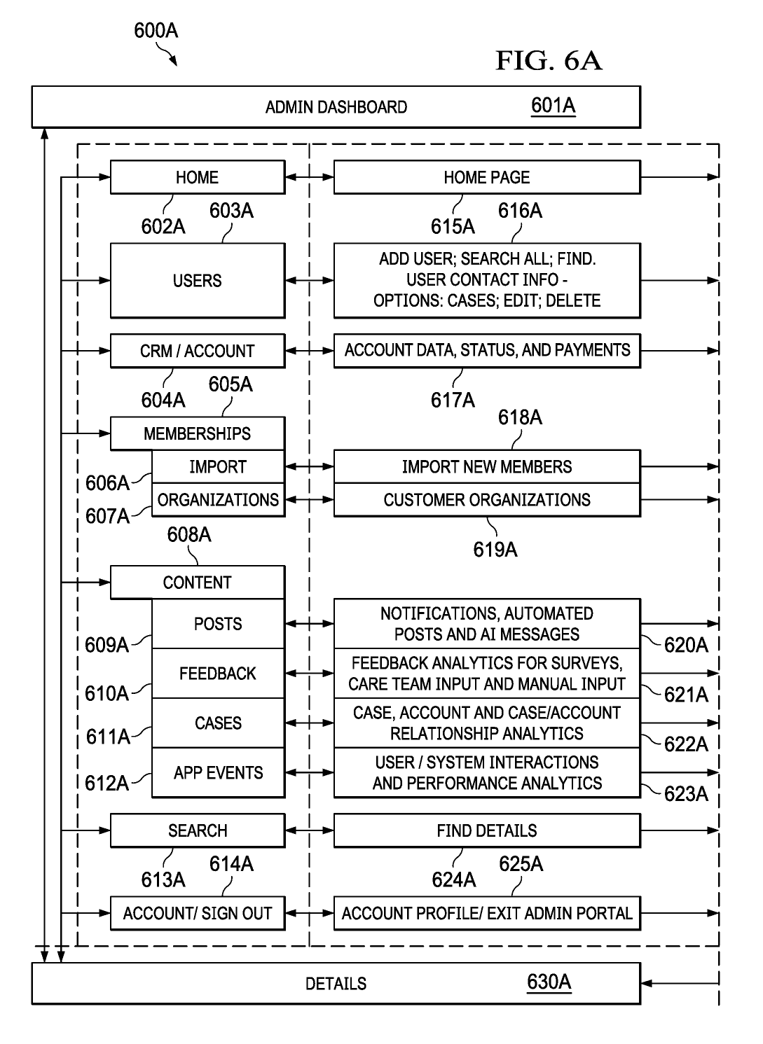

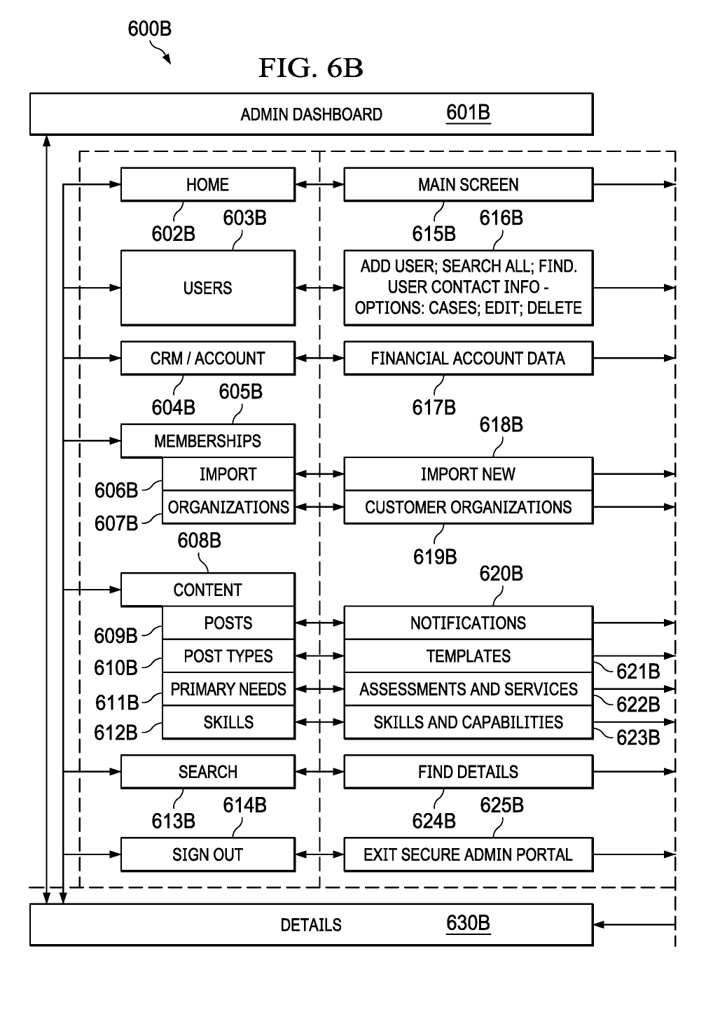

FIG. 6A is an illustrative example 600A of the interaction between admin dashboard 601A and tabs of the management system of the caregiver support platform. Embodiments with dashboards having differing selectable elements are envisioned within the invention to accommodate a user's needs. The admin dashboard 601A will display a plurality of selectable elements specific to the established rights of admin personnel. Admin has access to the totality of care cases as well as caregiver support platform details, information and resources. Tabs are the selectable elements on the dashboard that direct the admin to topic specific landing pages. The admin home page has a plurality of selectable elements, including, but not limited to Home 602A, Users 603A, Accounts 604A, Memberships 605A, Content 608A, Search 613A and Account/Sign out 614A.

The Home tab 602A of the management system of the caregiver support platform will direct the Admin user back the Home Page (or main screen) 615A with elements including a search function and tabs directing to admin functionalities. The Users tab 603A of the management system of the caregiver support platform directs to a list 616A of caregiver support platform users including personnel and caregiver users. Users can be added, deleted, edited or searched.

The CRM Accounts tab 604A of the management system of the caregiver support platform directs to a page 617A with account details and status, customer relationship management (CRM) processes and analytics, financial accounting processes, payment status and payment history, including payment status and payment history for all accounts.

Membership 605A directs to Import 606A and Organization 607A tabs. The Import page 606A is used to import new members 618A into the customer support platform database. The Organization tab 607A directs to a page 619A with details of customer organizations within the management system of the caregiver support platform, and has the option to add, edit, delete and search information.

Content 608A directs to tabs for case automation, and caregiver support platform analytics. The Posts tab 609A directs to automated notifications sent by the management system of the caregiver support platform to caregiver members and support platform personnel, including messages from the AI coaching assistant, surveys, notifications automatically generated based on input from users, and account notifications. The analytics are sent to the analytics engine of the management system of the caregiver support platform for processing to create dashboards and communication back to a Customer Organization, with user feedback, metrics for employee impact, and engagement of services by employees. The Feedback Analytic tab 610A directs to a page with analytics of information gathered from automated feedback surveys of caregiver members, care team feedback, and manually gathered feedback.

The Case Analytics tab 611A directs to a content page for account analytics, case analytics, case needs analytics, case discussion analytics, and Account/Case relationship analytics by the management system of the caregiver support platform. The App Event Analytics tab 612A directs to analyses of detailed and granular information about user and system interactions and performance by the management system of the caregiver support platform. The search tab 613A generates a match list 624A of text placed in the search window.

The Admin account tab 614A of the management system of the caregiver support platform directs to a dropdown option 625A to select the admin profile information or to sign-out. Admin personnel can edit the profile or sign-out of the admin portal. The tab can be customized with the Admin user's name.

FIG. 6B is an illustrative example 600B of another embodiment of the interaction between admin dashboard 601B and tabs of the management system of the caregiver support platform. Embodiments with dashboards having differing selectable elements are envisioned within the invention to accommodate a user's needs. The admin dashboard 601B will display a plurality of selectable elements specific to the established rights of admin personnel. Admin has access to the totality of care cases as well as caregiver support platform details, information and resources. Tabs are the selectable elements on the dashboard that direct the admin to topic specific landing pages. The admin home page has a plurality of selectable elements, including, but not limited to home 602B, users 603B, Accounts 604B, Memberships 605B and Content 608B, Search 613B and Sign out 614B.

The Home tab 602B of the management system of the caregiver support platform will direct the Admin user back the Home Page (or main screen) 615B with elements including a search function and tabs directing to admin functionalities. The Users tab 603B of the management system of the caregiver support platform directs to a list 616B of caregiver support platform users including details and contact information for personnel and caregiver users. Users can be added, deleted, edited or searched.

The CRM Accounts tab 604B of the management system of the caregiver support platform directs to a page 617B with account details and status, customer relationship management (CRM) processes and analytics, financial accounting processes, including payment status and payment history for all accounts.

Membership 605B directs to Import 606B and Organization 607B tabs. The Import page 606B is used to import new members 618BA into the customer support platform database. The Organization tab 607B directs to a page 619B with details of customer organizations within the management system of the caregiver support platform, and has the option to add, edit, delete and search information.

Content 608B directs to tabs for Posts 609B, Post types 610B, Primary needs 611B and Skills 612B. The Posts tab 609B directs to automated notifications sent by the management system of the caregiver support platform to caregiver members and support platform personnel, including messages from the AI coaching assistant, feedback surveys, notifications automatically generated based on input from users, and account status notifications. Post types 610B directs to templates 621B for notifications sent via the management system of the caregiver support platform. Notifications can be created, edited and prepared for distribution. Primary needs 611B directs to a page 622B with assessments of caregiver primary needs and services to meet those needs and the assessments may be used in the process of auto-assignment of support coaches with skills specific to the needs. Service and service groups are providers of services that will meet one or more of the needs in a care case, for example transportation or prescription services. Skills 612B directs to a page 623B with assessments of skills and capabilities of the support coaches and the assessments may be used in the auto-assignment process of support coaches with skills and capabilities to care cases requiring those skills.

The Search tab 613B generates a text match list of details placed in the search window 624B. The Sign out tab 614B allows for sign-out 625B from the Admin portal. The admin dashboards illustrated in FIGS. 6A and 6B are representations of the types of dashboards that can be used in the management system of the caregiver support platform. Selectable items on the dashboards represent types of tasks that may be performed by the dashboard user. Tasks and selectable items may be added or deleted depending on the display rights and/or needs of the user.

Case Automation. The management system of the caregiver support platform has a number of automated communication to support coaches and members. Some of these notifications utilize an AI coaching assistant support bot to maintain the human-like touch and a warm tone. Examples of automated notifications by the management system of the caregiver support platform, include but are not limited to, notifications to coaches when they have been assigned to a case, notifications to both support coaches and members when discussions, comments or files have been posted to the portal, notifications to the caregiver support team celebrating new organizations that offer the caregiver support service, and automated check-ins with caregiving members (e.g., Is everything ok?).

The AI coaching assistant of the management system of the caregiver support platform is used as a virtual assistant to continue to provide a personal touch to common communication to support the support coaches. The coaching assistant can ask for members for feedback, communicate alerts and updates and, assess possible emotional state/sentiment by reviewing key words and respond with support accordingly. The coaching assistant is intended to support coaches with certain types of automatable communication so support coaches can be more present with members, and to help maintain the human touch the support coaches work hard to establish with members. The coaching assistant can communicate with members by notifications through the member portal of the management system of the caregiver support platform or by email sent directly to the member. The avatar for the coaching assistant can display facial expressions appropriate to the content of the message.

Feedback Methods of Collection and Analytics. Feedback is gathered in several ways in the present invention. Input may be acquired directly from within the portal or by capturing organic feedback from members, clients and partners manually. One tool is an automated notification through the member portal of the management system of the caregiver support platform with a survey link included. Alternative methods for collecting feedback include Coaches collecting organic feedback provided by members without an auto-generated survey post to the member, engagement team members interviewing benefits managers at customer organizations or emailing members with the survey questions in the email.

For the automated notification, the feedback survey is posted by the AI coaching assistant of the management system of the caregiver support platform using the avatar artwork as the image icon. There will be a first post copy and subsequent post copy for the feedback survey. The First Post Copy is the copy used when requesting for feedback post is posted to a member for the first time. A first post copy may ask the member to take a 30-second survey about their experiences with the management system of the caregiver support platform. A launch button is provided for easy access. A subsequent feedback post by the AI coaching assistant through the member portal of the management system of the caregiver support platform might ask the member to take a follow-up survey at some later time. Again, a launch button is provided for ease of access.

When the “Take Survey” button is clicked, and pop up window appears and the user takes the survey within this modal. If the user chooses to not take the survey and clicks in the dark area surrounding the pop up window, the survey will close and should not register as completed. The survey should only register as completed once submitted. When the survey is completed and submitted the modal is closed and the user sees a “thank you” message posted from the AI coaching assistant. The feedback results are auto-posted by the management system of the caregiver support platform, however, the support coach has the option to manually post the feedback survey. If the support coach chooses to manually post the survey then the auto-post is canceled.

Care Team Feedback. As part of the Discussions process, the number of interactions between the support coaches and the rest of the Care Team are tracked by the management system of the caregiver support platform. At a defined point a Feedback survey is introduced into the Discussion. Each member of the Care Team will see the request. Each member of the Care Team that takes the survey will have their results captured independently. The Survey is designed to ask some quick, easy questions that gives the user a chance to provide input on whether the caregiver support platform and the Support coaches are meeting the member's expectations, by asking whether the service is helping the caregiving member to feel more confident, saving the caregiving member time, how helpful the support system has been, how valuable the support coach has been and how likely they would be to recommend the support service to others.

When the user has clicked the final Submit button, the results are captured to the database of the management system of the caregiver support platform and the user is presented with a “Thanks!” message from the AI Coaching Assistant. As the users experience progresses, additional follow up can be performed to ensure the proper quality of care is still being provided. The timing of the intervals is calculated to ask after a number of interactions and after a number of interactions slows down to avoid asking for feedback during a crisis event.

The information captured by the survey is stored to provide team feedback results. The fields tracked by the management system of the caregiver support platform are as follows: ID, confident, saved Time, helpful, valuable, recommend, feedback and createdAt.

Account Analytics. The account analytics of the management system of the caregiver support platform track all of the accounts in the system. The fields tracked are as follows: accountID, firstName, lastName, type, state, email, createdAt, updatedAt, heardFrom and additionalInfo.

Case Analytics. The case analytics of the management system of the caregiver support platform track all of the cases in the system. The fields we track are as follows: caseId, caseName, createdAt, updatedAt, lastActivity, casePriority, casePriorityDesc, coachLastActivity.

Case Needs Analytics. The case needs analytics of the management system of the caregiver support platform track all of the needs for each of the cases. This allows us to understand what the needs are holistically across all of the cases, or all of the needs of a particular Organization. The fields tracked are as follows: caseId, needName, needCount.

Case Discussion Analytics. The case discussion analytics of the management system of the caregiver support platform track the high level activity of the case. The information allows the system to track and understand interaction counts, types of communication and the ratio of communications from the Coaches to the rest of the Care Team. The fields tracked are: discussionId, totalPosts, totalCoachPosts, isUpload, totalAttachmetns, createdAt, updatedAt.

Account and Case Relationship Analytics. The accountCase analytics of the management system of the caregiver support platform tracks the relationships between the accounts and cases. The information provided provides metrics such as number of cases per account, and number of care team members per case. The fields tracked are: ID, case name, caseId, caseOwner, coachCoach, createdAt, updatedAt. The fields can be used to relate back to the other source analytics datasets Account and Case.

App Event Analytics. The App Events of the management system of the caregiver support platform provides detailed and granular information about user and system interactions and performance. These analytics allow us to see each system event (send an email, email sent, etc.) to user events (user opened email, etc.). These analytics allow data to be analyzed by the management system of the caregiver support platform to show user and support coach interactions (who did it, when they did it, how often they did it, etc.). These analytics track any and all changes to the case (for example, case payment status changes, Care team users added and removed, account ownership changes, case priority changes, case needs changes, etc.). All of this information allows for a holistic look at all cases to define patterns of usage that allow for predictions of coaching utilization levels, and support coach needs. This information allows for predictions of how the system will scale up in the continuing management of the number of coaches against case utilization.

The App Events fields tracked by the management system of the caregiver support platform are ID, event, createdAt, eventBy (user), eventType, accountID, caseID, pathname (for UI navigation tracking), subject, termsAcceptance, coachId, MandrillID, description, oldValue, newValue, userType, field, newOnwer and previousOwner.

All of the above Analytics are sent to the analytics engine of the management system of the caregiver support platform for processing to create dashboards and communication back to a Customer Organization, with user feedback, metrics for employee impact, and engagement of services by employees. Additionally the information is used to create dashboards by the management system of the caregiver support platform for the purposes of providing the executive team with a holistic view of the organization activities and performance.

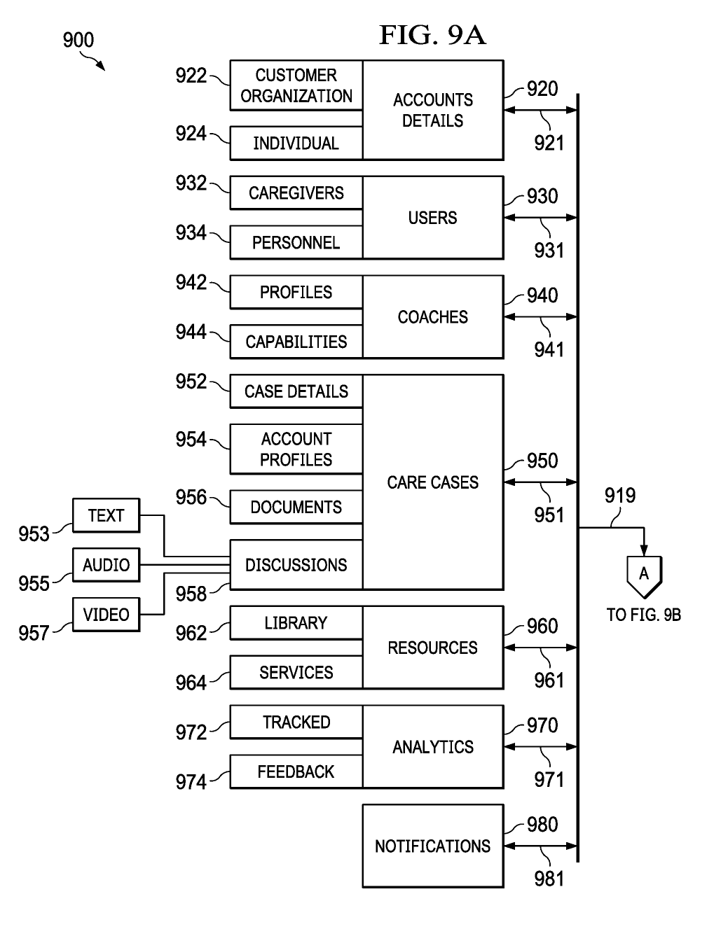

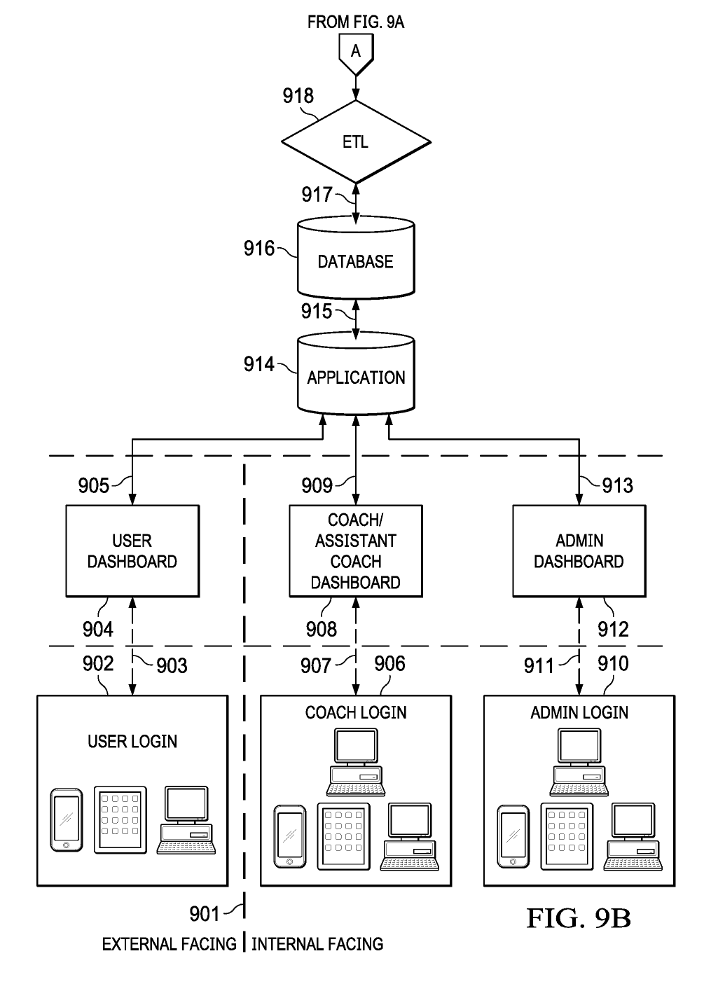

With regard to FIG. 9A-B, a caregiver support workflow 900 of the management system of the caregiver support platform including operations and data generation of users of the management system of the caregiver support platform is shown. The workflow 900 may include generating dashboard user interface data for caregiver users, support coach users and admin users to view on dashboard user interfaces utilizing the principles of the present invention and customized to the type of user. The workflow 900 may include caregiver support platform workflow 902, ETL procedures 918, database 916, and application server 914. The ETL procedures 918 may be configured to extract, transform, and load data generated by the management system of the caregiver support platform. The ETL procedures 918 may be software processes operating on the application server 914 and/or data collection systems 916, or may operate on a separate computing device. The application server 914 of the management system of the caregiver support platform may be configured to process and/or manage dashboard data for display on electronic devices, such as PCs, and mobile devices, such as laptops, tablets, and phones for displaying various dashboards. The caregiver support platform application is supported by the application server 914.

The dashboards may include admin dashboards 912 that access 913 the application server 914 and database 916 of the management system of the caregiver support platform. The dashboards may include support coach dashboards 908 that access 909 the application server 914 and database 916. The dashboards may include caregiver dashboards 904 that access 905 the application server 914 and database 916. It should be understood that additional and/or alternative dashboards may be available for display based on data collected and processed from the management system of the caregiver support platform. The dashboards may be interactive and dynamic for the caregiver user 902, support coach user 906 and admin user 910.

Admin dashboards 912 of the management system of the caregiver support platform enable admin personnel to view accounts, users, memberships and support platform content and analytics in a minimal number of “clicks,” such as three or four. The dashboards 912 provide access to account details for managing accounts, memberships, and support platform content and analytics.

Coach dashboards 908 of the management system of the caregiver support platform enable a support coach to answer questions related to assigned care cases in a minimal number of “clicks,” such as three or four. Moreover, the dashboards 908 may provide access to account details for managing care cases.

Caregiver user dashboards 904 of the management system of the caregiver support platform operate as a data access tools for caregiver and care team members. The dashboards enable a caregiver to quickly locate discussion topics, stored documents and resources suggested by the support coach in a minimal number of “clicks,” such as three or four.

Data of the management system of the caregiver support platform (920, 930, 940, 950, 960, 970, and 980) that was loaded into the database 916 would need to be transformed to application readable format by the management system of the caregiver support platform. The conversion or the extract load transform (ETL) process 918 would be done by the management system of the caregiver support platform using computer programming and transformational formulae. Communication lines are represented by arrows (917, 919, 921, 931, 941, 951, 961, 971, & 981).

The caregiver support platform data that was fed into the application server 914 of the management system of the caregiver support platform by the caregiver, support coach or admin user is loaded to a database 916 that would reside on a cloud or central setting. This data would then be converted through a variety of transformational formulae and ETL procedures 918 to be readable by the management system of the caregiver support platform application and displayed out via interactive user interface dashboards (904, 908 & 912) on the management system of the caregiver support platform.

The management system of the caregiver support platform Application server 914 is where the computer program or the application resides. The user would be interacting with the application in viewing the user interfaces or in this case the dashboards. This is an application that is hosted via the web. The data transformed by the management system of the caregiver support platform using transformational formulae or programming language, would then be displayed to the user on the user interface dashboards (904, 908 & 912).

Data for the management system of the caregiver support platform includes a number of data sets and subsets that are accessible based on a user's established rights. Account details data 920 has subsets for customer organizations 922, and individual accounts 924. User data 930 has subsets for caregivers 932 and personnel 934. Coach data 940 has subsets for coach profiles 942 and coach skills and capabilities 944. Care Case data 950 has subsets for case details 852, account profiles 954, Documents 956, Discussions 958. Discussion data can be written text 953, audio files 955 or video files 957. Discussion data also includes automated notifications that are posted to a care case. Resource data 960 has subsets for knowledge library 962 and provider services 964. Analytics data 970 includes data derived from risk assessment surveys and feedback surveys taken by users and has subsets for tracked data 972 acquired from system interactions and, feedback data 974 from surveys including manual input, and Notifications data 980 is derived from the automated messaging system on the management system of the caregiver support platform.

Communication Control. There are times where certain information is privileged for specific parties. The management system of the caregiver support platform supports the ability for the support coach and owner to choose who gets to see specific messages and files that are added to the portal. These can be set at the moment of creation or adjusted once added to the portal. A user's established rights will inform what selectable items are presented via the dashboard and what information is visible to the user.

Firebox. There is a set of core documentation that caregivers should have in place to provide the best care for their loved ones. The caregiver support platform educates members on what those documents are, and guides them through finding them, filling out necessary forms and storing the documents in a secure area on the portal of the management system of the caregiver support platform where they can be accessed on both desktop and mobile devices at the moment the documents are needed.

An overview of the management system and method of supporting the caregiver support platform includes the following process steps:

Contact a User by email generated by a marketing platform program (for Customer Organization employees) via the management system of the caregiver support platform. The User clicks a link in an email.

Access by the user of the company website of the management system of the caregiver support platform by clicking the link in the email (for Customer Organization employees), or an individual User may direct access the company website by typing in the web address (URL) or locating the website following an internet search.

Access Risk Assessment Survey by the user clicking a link on company webpage. The User answers decision tree type questions and responses are collected by a data collection application of the management system of the caregiver support platform.

Submit the survey and data is analyzed by data collection application of the management system of the caregiver support platform and a risk assessment level is displayed to the User.

The Create Account button is activated by the User initiating survey data to run through a script transforming and transferring the data to the management system of the caregiver support platform.

An Account is created by the user in the management system of the caregiver support platform. Upon creation of an account in the Platform, an individual User will be prompted for payment information. If the User is an employee of a Customer Organization with an “Organization Paid” contract, the User will not be prompted for any payment. Contract terms and conditions are stored in a Customer Relationship Management (CRM) system of the management system of the caregiver support platform.

The CRM is used to track contact information, tasks, activities and contract information for the customer associated with the account of the management system of the caregiver support platform. The information is shared via API with the management system of the caregiver support platform application's database upon case creation and on an ongoing basis to make sure the account is paid. If a case is created on the Customer Organization application, a secondary “Household” account is created underneath the Customer Organization account so the management system of the caregiver support platform can track the name of the case, member information for the case owner, case creation history, and other information pertaining to the case.

A Case is created n the management system of the caregiver support platform. The User (individual or employee) is prompted to provide information about the loved one needing care and the biggest caregiving challenge. The User is prompted to create and confirm a password to access the account.

Upon Case Creation, the User enters the management system of the caregiver support platform and receives a welcome message confirming successful account creation. The User's response to the biggest caregiving challenge is saved to the “notes” section on the internal facing Coaching Portal of the management system of the caregiver support platform.

In one embodiment, an assistant coach of the management system of the caregiver support platform receives an email that a case been created and needs a Coach to be assigned. The assistant coach can then login to the Coaching Portal to review the notes and assign a Coach. The Assistant Coach can also click the “Coaches” tab on the top of case notes screen where they can then select a Coach from the Caregiver Support team to assign to the case. When the Assistant coach has assigned a Coach to the case, the Coach receives an email with the assignment and the User receives an email notifying them of the contact information for the Coach to the case. The User view in the management system of the caregiver support platform will show that the Coach has been successfully added and the contact information for the Coach will be visible. The foregoing description of embodiments of the invention has been presented only for the purpose of illustration and description and is not intended to be exhaustive or to limit the invention to the precise forms disclosed. Numerous modifications and adaptations thereof will be apparent to those skilled in the art without departing from the spirit and scope of the present invention.

We claim:

1. A management system for a caregiver support platform comprising:
a computer processor having subprograms operating on the computer processor, and said computer processor coupled to one or more input/output devices, one or more input/output ports for receipt and transmission of data, requests, replies and commands, and a temporary memory for storage of data including caregiver support platform data, caregiver support platform resources, and administrative data;
one or more database memory coupled to said computer processor, said one or more database memory stores data and commands associated with operation of the subprograms including caregiver account data, caregiver support platform data, caregiver support platform resources, and administrative data;
an evaluation subprogram operated through the management system that executes a survey program with a first user to assess the first user's present status and potential risk of becoming a caregiver to another person, said evaluation subprogram assigns a first caregiver level to said first user based on the first user's input to said survey program and said evaluation subprogram transfers data input by the first user in response to said survey program into said computer processor and database memory;
an account creation subprogram operated on the management system that executes an account creation program to create a first care case on said management system associated with the first user based on the first caregiver level assigned to said first user, said account creation subprogram requesting and receiving information from the first user to be associated with the first user's account, and said account creation subprogram maintains information that can support the first user in the first care case;
a coaching subprogram operated on said management system that executes a coaching program that can be accessed by a support coach to support the entry and maintenance of data information and resources from the management system that can assist the first user; and,
a notification system subprogram operated on said management system that transmits messages to said first user based on requests, commands, responses or events related to the first user.

2. The management system of claim 1 further comprising:
an administrative subprogram operated on said management system that executes an administrative program to input and maintain data relating to the first user, including information and resources relating to the first user's status as a caregiver.

3. The management system of claim 1 further comprising:
an interactive dashboard display driven by said management system having customized displays of information directed to said first user, one or more other users, one or more support coaches, or one or more administrative personnel.

4. The management system of claim 1 wherein said management system allows access to information maintained on the management system and displayed on the interactive dashboard display based on display rights associated with said first user, one or more other users, one or more support coaches, or one or more administrative personnel.

5. The management system of claim 1 wherein said evaluation subprogram operated on the management system executes the survey program through an external facing portion of the management system.

6. The management system of claim 1 wherein said evaluation subprogram operated on the management system provides the first user with an option to create an account after completion of the survey and evaluation.

7. The management system of claim 1 wherein said account creation subprogram operated on the management system communicates to the first user through an external facing portion of the management system to create said first user's account having said first care case associated with the first user as a primary caregiver and account owner.

8. The management system of claim 1 wherein said account creation subprogram operated on the management system enters data from the first user as a primary caregiver on a caregiver portal, said data being received into said management system.

9. The management system of claim 1 wherein said coaching subprogram operated on the management system communicates to the first user through an internal facing portion of the caregiver support platform to support the input of data into the support coach portal, access data, information and resources maintained on the management system, and output data and information from the management system.

10. The management system of claim 1 further comprising:
an analytics protocol operated on the management system that analyzes the first user data input for the first care case account established for the first user and maintains personnel data related to the support coach assigned to the first user.

11. The system of claim 1, wherein said support coach assigned to the first user accesses data related to the first user, caregiver support platform information, and resources to prepare a care plan specific to the needs of the first user and provide one or more caregiver support coaching sessions to the first user with a transmission of output information to the first user.

12. The support system of claim 11, wherein the first care case can be accessed by the first user, and includes data and information including the care plan prepared by the support coach, audio and video discussions of coaching sessions with the first user, notifications from the management system, and resources made available to the first user from caregiver support system data repository.

13. The system of claim 1, wherein the external facing and internal facing subprograms may be accessed from, or input into, the computer processor using a desktop computer, mobile phone, intelligent pad device, or other personal communication device.

14. The system of claim 1, wherein upon account creation, the notification system subprogram transmits an automated message to the first user as the primary caregiver confirming the establishment of an account, an automated message to one or more support coaches assigned to the first user's care case, and an automated message to one or more administrative personnel reporting the first user's caregiver account creation.

15. The system of claim 1, wherein a care team is established that comprises the first user as the primary caregiver, one or more support coaches, and one or more additional caregivers authorized by the primary caregiver.

16. The system of claim 15, wherein the persons assigned to the care team are authorized by the first user as the primary caregiver to access the care case using management system portal to input documents, discussions, participate in coaching sessions and review stored coaching sessions.

17. The system of claim 1, further comprising an analytics engine to track, analyze, and report caregiver support platform interactions and events, survey responses, caregiver accounts, care case needs and discussions, and account and case relationships.

18. A method of operating a management system for a caregiver support platform comprising the steps of:
providing a computer processor having one or more input/output devices, one or more input/output ports for receipt and transmission of data, requests, replies and commands, and a temporary memory for storage of data including caregiver support platform data, caregiver support platform resources, and administrative data;
providing one or more database memory coupled to said computer processor, said one or more database memory stores data and commands associated with operation of the subprograms including caregiver account data, caregiver support platform data, caregiver support platform resources, and administrative data;
operating subprograms on the computer processor, and said subprograms include an evaluation subprogram, an account creation subprogram, a coaching subprogram, and a notification subprogram,
said evaluation subprogram operated through the management system that executes a survey program with a first user to assess the first user's present status and potential risk of becoming a caregiver to another person, said evaluation subprogram assigns a first caregiver level to said first user based on the first user's input to said survey program and said evaluation subprogram transfers data input by the first user in response to said survey program into said computer processor and database memory;
said account creation subprogram operated on the management system that executes an account creation program to create a first care case on said management system associated with the first user based on the first caregiver level assigned to said first user, said account creation subprogram requesting and receiving information from the first user to be associated with the first user's account, and said account creation subprogram maintains information that can support the first user in the first care case,
said coaching subprogram operated on said management system that executes a coaching program that can be accessed by a support coach to support the entry and maintenance of data information and resources from the management system that can assist the first user with their caregiving of another person; and,
said notification system subprogram operated on said management system that transmits messages to said first user based on requests, commands, responses or events related to the first user.

19. The method of operating a management system of claim 18 further comprising the step of:
operating an administrative subprogram on said management system that executes an administrative program to input and maintain data relating to the first user, including information and resources relating to the first user's status as a caregiver.

20. The method of operating a management system of claim 18 further comprising the step of:
providing an interactive dashboard display driven by said management system having customized displays of information directed to said first user, one or more other users, one or more support coaches, or one or more administrative personnel.

21. The method of operating a management system of claim 18 further comprising the step of:
allowing access to information maintained on the management system and displayed on the interactive dashboard display based on display rights associated with said first user, one or more other users, one or more support coaches, or one or more administrative personnel.

22. The method of operating a management system of claim 18 wherein said evaluation subprogram operated on the management system executes the survey program through an external facing portion of the management system.

23. The method of operating a management system of claim 18 wherein said evaluation subprogram operated on the management system provides the first user with an option to create an account after completion of the survey and evaluation.

24. The method of operating a management system of claim 18 wherein said account creation subprogram operated on the management system communicates to the first user through an external facing portion of the management system to create said new caregiver account having said care case associated with the first user as a primary caregiver and account owner.

25. The method of operating a management system of claim 18 wherein said account creation subprogram operated on the management system enters data from the first user as a primary caregiver on a caregiver portal, said data being received into said management system.

26. The method of operating a management system of claim 18 wherein said coaching subprogram operated on the management system communicates to the first user through an internal facing portion of the caregiver support platform to support the input of data into the support coach portal, access data, information and resources maintained on the management system, and output data and information from the management system.

27. The method of operating a management system of claim 18 further comprising the step of:
operating an analytics protocol on the management system that analyzes the first user data input for the care case account established for the first user and maintains personnel data related to the support coach assigned to the first user.

28. The method of operating a management system of claim 18 wherein said support coach assigned to the first user accesses data related to the first user, caregiver support platform information, and resources to prepare a care plan specific to the needs of the first user and provide one or more caregiver support coaching sessions to the first user with a transmission of output information to the first user.

29. The method of operating a management system of claim 28 wherein the caregiver account created for the first user can have associated data and information including the care plan prepared by the support coach, audio and video discussions of coaching sessions with the first user, notifications from the management system, and resources made available to the first user from caregiver support system data repository.

30. The method of operating a management system of claim 18 wherein the external facing and internal facing subprograms may be accessed from, or input into, the computer processor using a desktop computer, mobile phone, intelligent pad device, or other personal communication device.

31. The method of operating a management system of claim 18 wherein upon account creation, the notification system subprogram transmits an automated message to the first user as the primary caregiver confirming the establishment of an account, an automated message to one or more support coaches assigned to the first user's care case, and an automated message to one or more administrative personnel reporting the first user's caregiver account creation.

32. The method of operating a management system of claim 18 wherein a care team is established that comprises the first user as the primary caregiver, one or more support coaches, and one or more additional caregivers authorized by the primary caregiver.

33. The method of operating a management system of claim 32 wherein the persons assigned to the care team are authorized by the first user as the primary caregiver to access the care case using management system user portal to input documents, discussions, participate in coaching sessions and review stored coaching sessions.

34. The method of operating a management system of claim 18 further comprising the step of:
operating an analytics engine to track, analyze, and report caregiver support platform interactions and events, survey responses, caregiver accounts, care case needs and discussions, and account and case relationships.

35. A method of operating a management system for a caregiver support platform comprising the steps of:
providing a computer processor having one or more input/output devices, one or more input/output ports for receipt and transmission of data, requests, replies and commands, and a temporary memory for storage of data including caregiver support platform data, caregiver support platform resources, and administrative data;
providing one or more database memory coupled to said computer processor, said one or more database memory stores data and commands associated with operation of the subprograms including caregiver account data, caregiver support platform data, caregiver support platform resources, and administrative data;
operating an evaluation subprogram through the management system that executes a survey program with a first user to assess the first user's present status and potential risk of becoming a caregiver to another person, said evaluation subprogram assigns a first caregiver level to said first user based on the first user's input to said survey program and said evaluation subprogram transfers data input by the first user in response to said survey program into said computer processor and database memory;
operating an account creation subprogram on the management system that executes an account creation program to create a first care case on said management system associated with the first user based on the first caregiver level assigned to said first user, said account creation subprogram requesting and receiving information from the first user to be associated with the first user's account, and said account creation subprogram maintains information that can support the first user in the first care case;
operating a coaching subprogram on said management system that executes a coaching program that can be accessed by a support coach to support the entry and maintenance of data, information and resources from the management system that can assist the first user with their caregiving of another person;
operating a notification system subprogram on said management system that transmits messages to said first user based on requests, commands, responses or events related to the first user;
operating an administrative subprogram on said management system that executes an administrative program to input and maintain data relating to the first user, including information and resources relating to the first user's status as a caregiver; and,
providing an interactive dashboard display driven by said management system having customized displays of information directed to said first user, one or more other users, one or more support coaches, or one or more administrative personnel.

36. The method of operating a management system of claim 35 further comprising the step of:
allowing access to information maintained on the management system and displayed on the interactive dashboard display based on display rights associated with said first user, one or more other users, one or more support coaches, or one or more administrative personnel.

37. The method of operating a management system of claim 35 wherein said evaluation subprogram operated on the management system executes the survey program through an external facing portion of the management system.

38. The method of operating a management system of claim 35 wherein said evaluation subprogram operated on the management system provides the first user with an option to create an account after completion of the survey and evaluation.

39. The method of operating a management system of claim 35 wherein said account creation subprogram operated on the management system communicates to the first user through an external facing portion of the management system to create said new caregiver account having said care case associated with the first user as a primary caregiver and account owner.

40. The method of operating a management system of claim 35 wherein said account creation subprogram operated on the management system enters data from the first user as a primary caregiver on a caregiver portal, said data being received into said management system.

41. The method of operating a management system of claim 35 wherein said coaching subprogram operated on the management system communicates to the first user through an internal facing portion of the caregiver support platform to support the input of data into the support coach portal, access data, information and resources maintained on the management system, and output data and information from the management system.

42. The method of operating a management system of claim 35 further comprising the step of:
operating an analytics protocol on the management system that analyzes the first user data input for the care case account established for the first user and maintains personnel data related to the support coach assigned to the first user.

43. The method of operating a management system of claim 35 wherein said support coach assigned to the first user accesses data related to the first user, caregiver support platform information, and resources to prepare a care plan specific to the needs of the first user and provide one or more caregiver support coaching sessions to the first user with a transmission of output information to the first user.

44. The method of operating a management system of claim 43 wherein the caregiver account created for the first user can have associated data and information including the care plan prepared by the support coach, audio and video discussions of coaching sessions with the first user, notifications from the management system, and resources made available to the first user from caregiver support system data repository.

45. The method of operating a management system of claim 35 wherein the external facing and internal facing subprograms may be accessed from, or input into, the computer processor using a desktop computer, mobile phone, intelligent pad device, or other personal communication device.

46. The method of operating a management system of claim 35 wherein upon account creation, the notification system subprogram transmits an automated message to the first user as the primary caregiver confirming the establishment of an account, an automated message to one or more support coaches assigned to the first user's care case, and an automated message to one or more administrative personnel reporting the first user's caregiver account creation.

47. The method of operating a management system of claim 35 wherein a care team is established that comprises the first user as the primary caregiver, one or more support coaches, and one or more additional caregivers authorized by the primary caregiver.

48. The method of operating a management system of claim 47 wherein the persons assigned to the care team are authorized by the first user as the primary caregiver to access the care case using management system portal to input documents, discussions, participate in coaching sessions and review stored coaching sessions.

49. The method of operating a management system of claim 35 further comprising the step of:
operating an analytics engine to track, analyze, and report caregiver support platform interactions and events, survey responses, caregiver accounts, care case needs and discussions, and account and case relationships.
